(19) United States
(12) Patent Application Publication
(10) Pub. No.: US 2026/0041792 A1
(43) Pub. Date: Feb. 12, 2026

(54) NOVEL AAV3B CAPSID VARIANTS WITH ENHANCED HEPATOCYTE TROPISM

(71) Applicants: University of Florida Research Foundation, Incorporated, Gainesville, FL (US); The Trustees of Indiana University, Bloomington, IN (US); Cornell University, Ithaca, NY (US)

(72) Inventors: Sergei Zolotukhin, Gainesville, FL (US); Moanaro Biswas, Fishers, IN (US); Damien Marsic, Gainesville, FL (US); Jyoti Rana, Indianapolis, IN (US); Ype P. de Jong, Brooklyn, NY (US)

(73) Assignees: University of Florida Research Foundation, Incorporated, Gainesville, FL (US); The Trustees of Indiana University, Bloomington, IN (US); Cornell University, Ithaca, NY (US)

(21) Appl. No.: 18/993,282

(22) PCT Filed: Jul. 12, 2023
(86) PCT No.: PCT/US2023/070080

§ 371 Date: Jan. 10, 2025

Related U.S. Application Data
(60) Provisional application No. 63/388,633, filed on Jul. 12, 2022.

(51) Int. Cl. A61K 48/00; A61K 38/17; A61K 38/57; C07K 14/005; C12N 15/86

(52) U.S. Cl. CPC A61K 48/0058; A61K 38/1709; A61K 38/57; C07K 14/005; C12N 15/86; C12N 2750/14122; C12N 2750/14143

(57) ABSTRACT

Disclosed herein are recombinant AAV variant (e.g., variant serotype 3B (AAV3B)) capsid proteins and variant capsid protein-containing viral particles with enhanced ability to transduce hepatic cells. Viral particles containing these capsid variants are capable of evading neutralization by the host humoral immune response. The recombinant AAV3B variant proteins and viral particles disclosed herein were identified from a variant AAV3B capsid library that was engineered by making substitutions in only the variable regions of the capsid. Some embodiments of the AAV3B capsid variants disclosed herein comprise the AAV3B-V04 and the AAV3B-V05 variants. Compositions of these variant AAV particles are provided that are useful for transducing and delivering therapeutic transgenes to cells, such as liver cells, and thus treat diseases and disorders pertaining to these cells.

### RELATED APPLICATIONS

This application is a national stage filing under 35 U.S.C. 371 of International Patent Application Serial No. PCT/US2023/070080, filed Jul. 12, 2023, entitled “NOVEL AAV3B CAPSID VARIANTS WITH ENHANCED HEPATOCYTE TROPISM”, which claims the benefit under 35 USC 119(e) of U.S. Provisional Application No. 63/388,633, filed Jul. 12, 2022. The contents of these applications are incorporated herein by reference in their entirety.

### GOVERNMENT SUPPORT

This invention was made with government support under Grant No. HL131093, awarded by the National Institutes of Health. The government has certain rights in the invention.

### REFERENCE TO AN ELECTRONIC SEQUENCE LISTING

The contents of the electronic sequence listing (U119670103US01-SUBSEQ-PRW.xml; Size: 345,913 bytes; and Date of Creation: Sep. 29, 2025) are herein incorporated by reference in its entirety.

### BACKGROUND OF THE INVENTION

Adeno-associated virus (AAV) is a single-stranded DNA virus belonging to the Parvoviridae family (Muzyczka and Berns, 2001). AAV-derived vectors are promising tools for human gene therapy applications because of their absence of pathogenicity, low immunogenicity, episomal localization, and stable transgene expression. However, significant limitations to the clinical use of AAV are its promiscuity and its susceptibility to neutralization by human antibodies (Jeune et al., 2013). Both of these limitations are determined by nature of the amino acid residues exposed at the surface of the capsid. Therefore, major efforts aiming at developing useful and effective gene therapy vectors have been devoted to obtaining and studying capsid variants (Wu et al., 2006). The first approach was to study naturally occurring AAV isolates. So far, 13 serotypes have been formally characterized and hundreds of variant isolates have been sequenced. Additional capsid variation has been investigated through the generation of mosaics (viral particles made of capsid proteins from more than one serotype) (Hauck et al., 2003; Stachler and Bartlett, 2006; Gigout et al., 2005), chimeras (capsid proteins with domains from various origins) (Shen et al., 2007), and various substitutional or insertional mutants (Wu et al., 2000). However, the most significant advances are expected to result from directed evolution approaches through the development of capsid libraries.

The state of the art method for randomizing an AAV capsid-encoding genetic sequence was until recently error-prone polymerase chain reaction (PCR), which introduced randomly dispersed mutations throughout the roughly 730 amino acids that constitute the AAV capsid sequence. However, the error-prone PCR technique suffered from two key problems. First, random mutagenesis often installed mutations that were deleterious to capsid function, as only 0.01-1% of mutations were typically beneficial (see, e.g., Romero & Arnold (2009), Nat Rev Mol Cell Biol 10(12): 866-876 and Guo, Choe & Loeb (2004), Proc Natl Acad Sci USA 101(25): 9205-9210.). Second, the sheer number of PCR clones that needed to be generated to cover all combinations of multiple mutations within a single capsid by far exceeded the technical capabilities of the skilled artisan. For instance, it has been calculated that, to comprehensively randomize five residues within a 414 base pair fragment of the AAV2 capsid VP1 gene, an AAV library would have to comprise nearly 10¹¹ different clones to cover a single mutation at each site (see Maersch et al. (2010), Virology 397(1): 167-175).

The various strategies to generate capsid libraries that have been developed so far all suffer from sequence bias or limited diversity. Random display peptide libraries (Govindasamy et al., 2006) are limited to an insertion at one particular capsid location. Libraries generated using error-prone PCR contain a very small fraction of gene variants encoding proteins that can fold properly and assemble into a functional capsid, due to the randomness of the mutations. DNA shuffling and staggered extension processes are more efficient because they recombine naturally-occurring parental sequences and therefore are more likely to generate actual capsid variants. However, they can only recombine blocks of DNA as opposed to single nucleotide positions, which results in sequence bias (parental polymorphisms will tend to cluster together instead of being randomly distributed).

### SUMMARY OF THE INVENTION

The present disclosure provides adeno-associated virus (AAV) capsid variants, particles, and virions comprising capsid variants that exhibit enhanced transduction in hepatocytes. In certain embodiments, these capsid variants are capable of evading neutralization by host antibodies.

Accordingly, in some aspects, the present disclosure provides modified capsids of serotype 3B, also known as modified AAV3B capsids or AAV3B variants. In some aspects, the present disclosure provides the AAV3B-V04 capsid variant, or “V04.” In some aspects, the present disclosure provides the AAV3B-V05 capsid variant, or “V05.” V04 contains 9 amino acid substitutions, and V05 contains 21 substitutions, relative to wild-type AAV3B. In exemplary embodiments, the V04 capsid variant is provided.

The present disclosure is based, at least in part, on the rational generation of AAV capsid variant libraries through the introduction of motifs of novel mutations in the native capsid through mutagenesis and directed evolution. The present disclosure is further based on the screening of variants from amongst these libraries. The capsid variant V04 was undetectable in the original AAV library but gained a selective advantage following a bioinformatics analysis. According to the present disclosure, molecular evolution using a combinatorial library platform has generated capsid variants with high hepatocyte tropism and enhanced evasion of pre-existing AAV neutralizing antibodies.

The development of next-generation recombinant AAV (rAAV) particles or virions may dramatically reduce the number of viral particles needed for a conventional gene therapy regimen. In addition to having improved transduction efficiencies for various mammalian cells, the rAAV particles and virions prepared as described herein may be more stable, less immunogenic, and/or can be produced at much lower cost, or in a higher titer, than an equivalent wildtype viral vector prepared in conventional fashion.

In the practice of the present disclosure, native amino acids normally present in the sequence of a viral capsid protein, such as a wild-type capsid of serotype 3B, may be substituted by one or more non-native amino acids, including substitutions of one or more amino acids not normally present at a particular residue in the corresponding wild-type protein. In some embodiments, the amino acid substitutions in the disclosed capsid variants may be epistatic (interacting) with respect to one another. These amino acid substitutions may act synergistically on capsid binding and transduction behavior. In some embodiments, the amino acid substitutions comprise one or more motifs.

In some embodiments, the amino acid substitutions in the disclosed capsid variants confer upon particles and virions comprising these variants an enhanced ability to evade neutralizing antibodies of the host immune system. In some embodiments, the disclosed particles and virions have reduced seroreactivity. In some embodiments, the disclosed particles and virions are able to evade the humoral immune response, e.g., neutralizing antibodies, of a subject following their delivery into the subject. In particular embodiments, the subject is mammalian. The subject may be human. The subject may be a non-human primate.

Wild-type AAV is a small (˜26 nm), non-enveloped parvovirus. It packages a linear single-stranded DNA genome (˜4.7 kb), encoding genes necessary for replication (rep) and the viral capsid (cap), flanked by palindromic inverted terminal repeats (ITRs). Except for the ITRs which are essential, much of the viral DNA genome can be omitted for the purpose of transgene packaging and delivery, allowing for insertion of approximately 4.7 kb of foreign DNA, which altogether forms the transgene expression cassette⁶. In some cases, a self-complementary single strand duplex DNA can be packaged⁷, although this reduces the transgene capacity to less than half, and increases the risk of immune response⁸. Accordingly, in some embodiments, a naturally-occurring viral genome may be replaced with nucleic acids of interest (e.g., nucleic acids comprising transgenes or transgene expression cassettes) which becomes flanked by AAV ITRs, thereby producing an rAAV genome. In some embodiments, rAAV genomes may be packaged into rAAV particles. In some embodiments, rAAV particles of the present disclosure comprise one or more variant AAV3B capsid proteins (e.g., V04, V05, or other variant described in this application).

There are several challenges that impede the successful and broad use of rAAV gene therapy. The first major limitation to systemic or intramuscular administration of AAV is the presence of pre-existing neutralizing antibodies (NAb) against the vector capsid that can block cellular entry⁹. A majority of the human population are seropositive for AAV, mostly due to previous subclinical exposure to the wild-type virus¹⁰. This is a major exclusion criterion for prospective patients, as even very low titers of NAb in circulation can prevent vector entry and significantly limit effective gene transfer to the target organ. Pre-existing anti-AAV NAb do not affect rAAV injection into immune privileged sites such as the eye or brain. Luxturna® (voretigene neparvovec-rzyl), the FDA approved drug for treating inherited retinal disorders, can be successfully delivered into the eye using AAV serotype 2, which is seroprevalent in 40-70% of the human population¹⁰⁻¹². The impact of low titer NAb (<1:5), particularly on systemic gene transfer is not accurately known¹³⁻¹⁵. Using a different serotype is complicated because a pattern of cross reactivity commonly occurs between variants, such as between AAV2, AAV5 and AAV8¹⁶, or AAV1 and AAV6¹⁷, depending on the degree of homology between capsid protein sequences.

A second limitation is transduction efficiency of target cells. Based on specific receptor interaction and post-entry mechanisms, AAV serotypes differ in cell tropism as well as transduction efficiency in the cell type of choice¹⁸. A major challenge to gene therapy is that the functional gene may not transduce the target tissue in high enough numbers to provide therapeutic benefit. Increasing the rAAV dose in this case is not always effective, as a high viral load can induce detrimental capsid specific T cell immune responses to the transduced cell^{14, 19}.

These limitations cannot adequately be addressed by the current limited repertoire of naturally occurring AAV serotypes, and the isolation and characterization of novel variants is time consuming. The nature of exposed amino acid residues on the capsid surface largely determines receptor attachment, tissue transduction, and antigenicity²⁰. Therefore, the development of engineered AAV vectors designed either by rational modification of specific amino acids (rational mutagenesis), or in vitro or in vivo selection in the cell type of choice (directed evolution) is an attractive alternative^{21, 22}. These novel rAAV capsid vectors based on different serotypes have been shown to be superior at transducing the cell type of interest and, in some cases, evading pre-existing NAb in the host²³⁻²⁶ Rationally-generated AAV capsid libraries containing modified AAV capsids based on serotype 3B have been recently described. See International Patent Publication No. WO 2017/070476, published on Apr. 27, 2017, herein incorporated by reference. AAV3B capsid variants containing various combinations of mutations in the surface-exposed Y, S, and T residues have been generated, and an S633V+T492V mutant (AAV3B.ST, or AAV3-ST) was identified to possess enhanced capacity to transduce primary human hepatocytes in vitro. See Ling, C, et al., Mol Ther. 2014; 22: S2, incorporated herein by reference. Previous studies have shown that systemically delivered AAV3B-ST performed better than AAV5, AAV8, AAV9 and wt AAV3B in a human liver xenograft mouse model as well as non-human primate livers^{33, 43, 44} Another engineered chimeric AAV3B capsid variant, LK03, which was derived from an AAV shuffled library, is closely related to wt AAV3B, with only eight amino acid changes. LK03 (or AAV3-LK03) has shown strong tropism for human hepatocytes in humanized mouse livers²³, and is currently being evaluated in clinical trials for hemophilia A gene therapy (NCT03003533). While AAV8 and AAV5 serotypes are used for in several clinical trials for hepatic gene transfer, emerging data in non-human primates and in “humanized” chimeric liver mice engrafted with human hepatocytes indicate that engineered vectors on the AAV3B backbone show superior transduction efficiencies in human liver^{23, 33, 44.}

Unlike what would be expected from error-prone PCR, the novel mutations of the capsid variants of the present disclosure were not randomly or arbitrarily selected. In the present disclosure, rational mutagenesis and directed evolution strategies were combined to select for engineered AAV vectors derived from the AAV3B capsid backbone. For instance, in some embodiments, the presently disclosed engineered variants show improved tropism for human hepatocytes in vitro as compared to wild type (wt) AAV3B, along with AAV3B variants AAV3-ST and LK03. In some embodiments, the presently disclosed variants show improved human hepatocyte transduction in an in vivo liver chimeric mouse model. Furthermore, in some embodiments, the disclosed variants exhibit reduced seroreactivity to pre-existing NAb from both pooled human IVIg, as well as in individual serum samples tested from a hundred healthy human donors. In some embodiments, these variants exhibit such reduced seroreactivity relative to, e.g., wild-type AAV3B.

Certain embodiments of the modified AAV capsids, AAV particles, and AAV virions of the present disclosure include the second nucleotide sequence encoding an AAV Cap protein that differs from wildtype serotype 3 VP1 capsid protein at least at one amino position. In some embodiments, the at least one amino acid position that differs is preferably in a variable region (VR), and may be in variable regions 1, 4, 5, 6, 7, or 8 (VR-I, VR-IV, VR-V, VR-VI, VR-VII, VR-VIII) and combinations thereof.

The present disclosure provides variant recombinant adeno-associated virus (rAAV) serotype 3B (AAV3B) capsid protein comprising any of the following sets of sequences and/or substitutions in the wild-type of AAV3B VP1 sequence of SEQ ID NO: 1. Certain aspects of the modified AAV capsids, AAV particles, and AAV virions of the present disclosure include VR-IV encoding amino acid sequence SNAX₁GTX₂X₃X₄X₅X₆LX₇ (SEQ ID NO: 7) where X₁ is independently G or S; X₂ is A; X₃ is independently N or G; X₄ is independently T or M; X₅ is independently R or N; X₆ is independently R or G; and X₇ is M. In certain embodiments, X₁ is S; X₃ is G; X₄ is T; X₅ is R; and X₆ is G.

In certain embodiments, the VR-IV of the modified capsid encodes an amino acid sequence motif of SNAGGTANMNRLM (SEQ ID NO: 3). In certain embodiments, the VR-IV of the modified capsid encodes an amino acid sequence motif of SNASGTAGTRGLM (SEQ ID NO: 8) in VR IV.

Certain aspects of the modified AAV capsids, AAV particles, and AAV virions of the present disclosure include VR-V encoding amino acid sequence X₁₁X₁₂X₁₃X₁₄NNNSNFPWTAASX₁₅ (SEQ ID NO: 19) where X₁₁ is independently I or T; X₁₂ is independently A or P; X₁₃ is independently N, S or G; X₁₄ is independently D or Q; and X₁₅ is independently K or T. In certain embodiments, the VR-V of the modified capsid encodes an amino acid sequence motif of TIDTQNNNSNFPWTAASK (SEQ ID NO: 4).

Certain aspects of the modified AAV capsids, AAV particles, and AAV virions of the present disclosure include VR-VI encoding amino acid sequence KDDX₁₆X₁₇X₁₈ where X₁₆ is independently E or D; X₁₇ is independently E or D; and X₁₈ is independently K or R. In certain embodiments, X₁₆ is D; X₁₇ is D; and X₁₈ is R. In certain embodiments, the VR-VI of the modified capsid encodes KDDEER (SEQ ID NO: 9).

Certain aspects of the modified AAV capsids, AAV particles, and AAV virions of the present disclosure include VR-VIII encoding amino acid sequence QX₃₀X₃₁X₃₂X₃₃X₃₄PTX₃₅RX₃₆VX₃₇X₃₈ (SEQ ID NO: 40) where X₃₀ is independently S or N; X₃₁ is independently S or G; X₃₂ is independently N or R; X₃₃ is independently T or D; X₃₄ is independently A or N; X₃₅ is independently T or F; X₃₆ is independently T or D; X₃₇ is independently N or Q; and X₃₈ is independently H or D. In certain embodiments, X₃₀ is S; X₃₁ is S; X₃₂ is N; X₃₃ is T; X₃₄ is A; X₃₅ is F; X₃₆ is T; X₃₇ is N; and X₃₈ is D. In certain embodiments, the VR-VIII of the modified capsid encodes an amino acid sequence motif of QGAEASNVEVGK (SEQ ID NO: 6).

In some embodiments, the disclosed AAV3B capsid variants contain a mutated VR IV sequence and wild-type VR V, VR VI, VR VII, and/or VR VIII sequences.

In some embodiments, the rAAV particles and rAAV virions of the present disclosure are contacted, transduced, and/or incorporated into at least one host cell. Examples of suitable host cells are mammalian cells including human host cells, including, for example, blood cells, stem cells, hematopoietic cells, CD34 cells, liver cells, cancer cells, vascular cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a mammal for which viral-based gene therapy is contemplated. In some embodiments, the host cells are liver cells (hepatocytes). In some embodiments, the host cells are human liver cells.

AAV particles, rAAV particles, and rAAV virions comprising the exemplary AAV3B variants of the present disclosure may include the particles and virions as incorporated or transduced into at least one host cell. Examples of suitable host cells include human hepatocytes, e.g., hepatocellular carcinoma cell lines HUH-7 and HepG2, murine hepatocytes, e.g., H2.35, HEK293 (embryonic kidney) cells, HeLa cells, Cos cells, U87 cells, KB cells, and Vero cells. In certain embodiments, the modified AAV3B particles and virions of the present disclosure are incorporated into HUH-7, H2.35 and/or HepG2 cells. In particular embodiments, virions comprising the V04 and V05 variants are incorporated into HUH-7, H2.35 and/or HepG2 cells.

In some embodiments, the AAV particles and virions of the present disclosure are produced in a host cell in a method comprising contacting one or more host cells with a nucleotide acid encoding at least one molecule providing helper function. In some embodiments, a third nucleic acid (e.g., a helper nucleic acid) may be a polynucleotide comprising sequences derived from an adenovirus helper virus or a herpes helper virus (e.g. recombinant HSV1). In particular embodiments, the helper nucleic acid comprises sequences that is derived from adenovirus, e.g., Ad5.

In some aspects, the disclosure provides methods of selecting tissue-specific or cell-specific variants of AAV virions including (a) introducing a plurality of AAV virions into target tissues or cells; (b) allowing sufficient time to elapse to propagate additional virions; and (c) isolating the virions. Steps (a) through (c) may be repeated one or more times to enrich for a tissue-specific (e.g., hepatic tissue-specific) or cell-specific variant. Such enriched variants exhibit a higher target tropism for the target tissues or cells as compared to AAV serotype 3.

An embodiment of the AAV virions of the present disclosure includes (a) a first nucleotide sequence encoding at least one therapeutic molecule; (b) a second nucleotide sequence comprising a regulatory sequence; (c) a third nucleotide sequence comprising a first AAV terminal repeat (e.g., from serotype 3); (d) a fourth nucleotide sequence comprising a second AAV terminal repeat (e.g., from serotype 3); and (e) a capsid comprising at least one AAV Cap protein that differs from wildtype serotype 3 at least at one amino acid position. In some embodiments, the first nucleotide sequence is operably linked to the second nucleotide sequence and the first and second nucleotide sequences are interposed between the first and second AAV terminal repeat to form a transgene, and the resulting transgene is packaged within the capsid. Examples of suitable regulatory sequences include, but are not limited to, promoters and enhancers, e.g., a tissue specific promoter. Examples of suitable therapeutic molecules include, but are not limited to, polypeptides, peptides, antibodies, antigen binding fragments, growth factors, cytokines and other small therapeutic proteins, and any combination thereof.

In some aspects, the present disclosure provides methods for treating a disease or disorder. In some embodiments, such methods may comprise administering an effective amount of an AAV virion of the present disclosure. In some embodiments, the disease or disorder is Alpha-1 Antitrypsin Deficiency or Transthyretin-Related Familial Amyloid Polyneuropathy.

The following drawings form part of the present specification and are included to demonstrate certain aspects of the present invention. The invention may be better understood by reference to the following description taken in conjunction with the accompanying drawings, in which like reference numerals identify like elements.

### BRIEF DESCRIPTION OF THE DRAWINGS

FIG. 1 shows the wildtype (WT) nucleotide sequence (bottom rows) and corresponding WT amino acids (top rows, bold font) of AAV3B capsid gene and capsid protein, respectively. Degenerate positions within each variable region (VR) diversified in AAV serotype 3 capsid library (A3CL) are highlighted. The degenerate nucleotide positions (in IUPAC code) encoded by synthetic oligonucleotides are shown in italics below the WT sequence.

FIG. 2 shows the nucleotide sequence of the synthetic fragment A3CL as designed. The degenerate nucleotide positions (in IUPAC code) are underlined. The overlap stretches of the synthetic DNA and the plasmid vector backbone are highlighted.

FIG. 3 shows the amino acid sequence of AAV3B VP1. Degenerate positions are labeled by X and underlined.

FIG. 4 shows the amino acid sequences of the AAV3B (A3CL) VRs encompassing WT AAV3B VP1 capsid residues 259-600. WT sequences are shown in black, degenerate residues—in italics. Not modified conservative residues between VRs are not shown. VRs borders are indicated by vertical lines.

FIG. 5 is a flowchart illustrating design and construction of AAV3B (A3CL) combinatorial capsid libraries ABC and D.

FIGS. 6-9 are photographs of agarose gels showing products of PCR reactions as per Example 2.

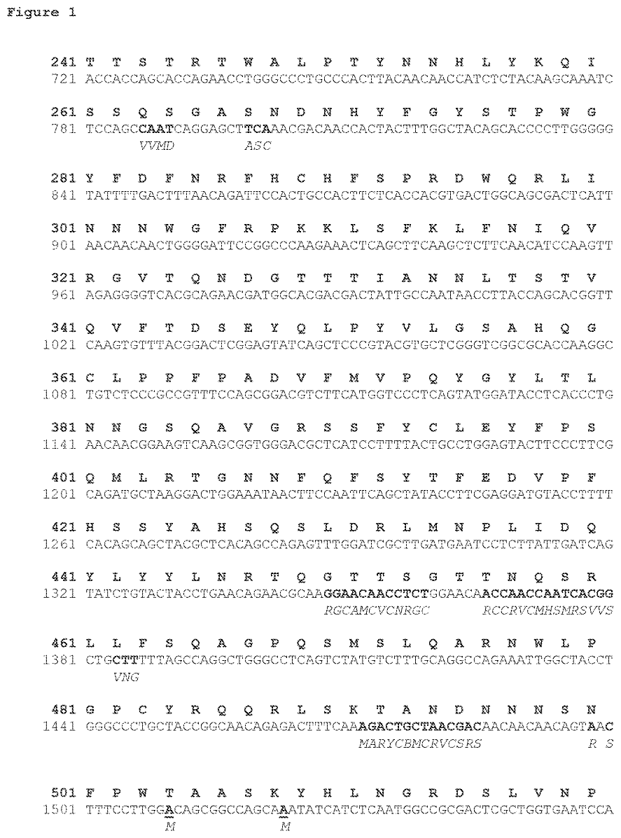

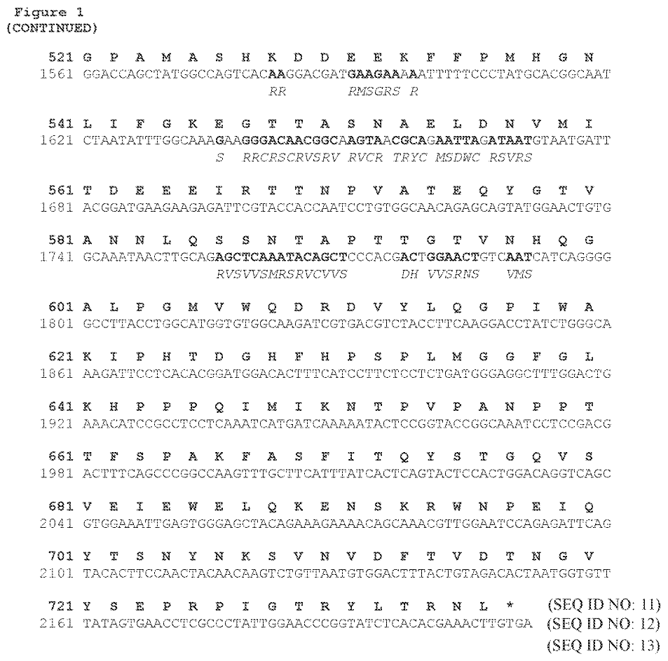

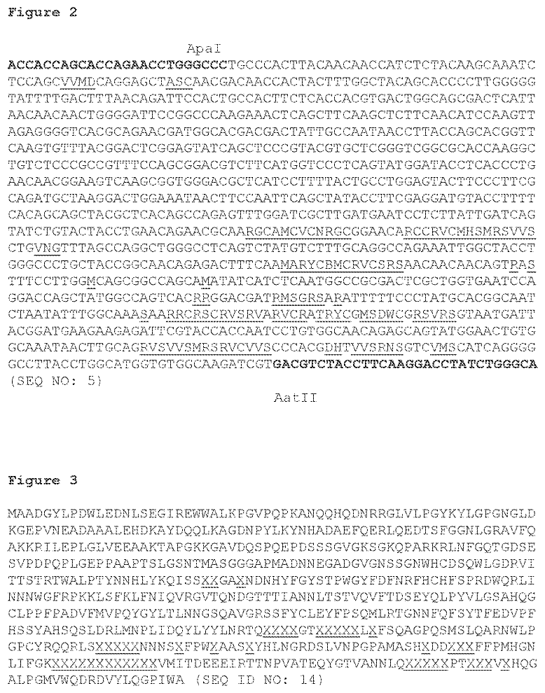

### DETAILED DESCRIPTION OF THE INVENTION

AAV-derived viral particles (e.g., recombinant AAV (rAAV) particles) are promising tools for human gene therapy applications because of reduced pathogenicity compared to other viral vectors, episomal localization, and stable transgene expression. rAAV viral particles show huge promise for the delivery of therapeutic genes to the liver. Improving the transduction efficiency of rAAV particles having tropism for hepatic cells would be of great benefit to disease of the liver, including Alpha-1 Antitrypsin Deficiency and Transthyretin-Related Familial Amyloid Polyneuropathy. AAV virions of serotypes 3 and 3B have been demonstrated to possess tropism for liver cells. See Li et al., Mol Ther. 2015; 23(12): 1867-1876 and Glushakova, L G et al., Mol Genet Metab. 2009; 98: 289-299, each of which are herein incorporated by reference. This in part due to AAV3B's use of human hepatocyte growth factor receptor (huHGFR) as a cellular coreceptor.

The tissue tropism and transduction efficiency of AAV particles (and recombinant particles thereof) is determined by the nature of amino acid residues exposed at the surface of the capsid (Wu et al., J Virol. 2006, 80(22):11393-7, herein incorporated by reference). Therefore, manipulating the amino acids of the capsid proteins provides an opportunity to fine-tune the tissue tropism of the particle and also improve transduction efficiency. However, certain manipulations, e.g., substitutions of amino acids, of the capsid protein can cause it to mis-fold or not form a capsid at all. To circumvent issues of protein mis-folding and capsid mis-forming, the recombinant AAV3B (rAAV3B) variant proteins and viral particles disclosed herein were identified from a variant AAV3B capsid library that was built by making substitutions in only the variable loops of the capsid protein. Herein, “variable loops” are also referred to as “variable regions”. AAV3B has 9 variable regions, numbered from VRI to VRIX.

It was previously shown that pre-existing neutralized antibodies (NAb) against AAV3B are relatively lower (48% of animals with detectable NAb) as compared with AAV8 (≥75% of animals positive for AAV8 NAb) in non-human primates (see Li et al., Mol Ther. 2015). Screening of an AAV3B capsid library in a mouse model led to the identification of AAV3B capsid variants that possess enhanced efficiency to transduce hepatic cells compared to the transduction efficiency of wild-type AAV3B capsid proteins.

Accordingly, in some embodiments, the virions disclosed herein (e.g., V04 and V05 virions) may demonstrate reduced seroreactivity relative to a wild-type AAV3B capsid, or relative to another AAV3B variant capsid. In some embodiments, the virions disclosed herein may evade neutralizing antibodies (Nab) of host liver cells in vivo, e.g., in a subject, such as a primate (e.g., a human or a non-human primate). In some embodiments, the disclosed virions provide an about 1.5-fold, a 2-fold, a 2.5-fold, a 3-fold, a 3.2-fold, a 3.5-fold, a 4-fold, a 5-fold, a 6-fold, a 10-fold, a 12-fold or a 15-fold decrease in seroreactivity to neutralizing anti-AAV (e.g., anti-AAV3) antibodies in the subject, relative to a recombinant AAV3B virion comprising wildtype AAV3B capsid proteins. In some embodiments, the virions provide an about 2-fold decrease in seroreactivity to neutralizing anti-AAV antibodies in the subject, relative to a recombinant AAV3B virion comprising wildtype AAV3B capsid proteins.

Accordingly, in some embodiments, provided herein are mutants (e.g., variants) of wild-type AAV3B capsids, compositions of such particles and methods of using these compositions to transduce hepatic cells, exhibit reduced sero-reactivity, and/or evade a host humoral immune response.

Reduced seroreactivity and evasion of NAb in subjects may be measured by any method known in the art. In some embodiments, the degree of reduced seroreactivity and/or evasion of NAb is evaluated in vivo in human sera by measuring the differential expression of a protein encoded in the rAAV genome (e.g., an rAAV genome comprising a transgene) (which indicates the degree of transduction of that protein) of an administered virion in a sample obtained from a subject that had been administered the virions. In other embodiments, degree of reduced seroreactivity and/or evasion of NAb is evaluated in vitro by pre-incubating an rAAV virion encoding a protein with pooled IVIg, transducing one or more cells (e.g., human cells) with the pre-incubated virions, and measuring the differential percent of transduction (i.e., % expression of encoded protein) by flow cytometry between samples.

In some aspects, the present disclosure provides variants of the wild-type AAV3B capsid. The wild-type AAV3B capsid, VP1 region is set forth as SEQ ID NO: 1, below. In some embodiments, the variants, or modified capsids, of the present disclosure have an amino acid sequence essentially (e.g., comprising one or more differing positions) as set forth in SEQ ID NO: 1. In certain embodiments, the modified AAV capsid is truncated by 1, 2, 3, 4, 5, 6, 7, 8, 9, 10, 11, 12, 13, 14, 15, or 15-20 amino acids relative to the wild-type AAV3B VP1 sequence of SEQ ID NO: 1.

(SEQ ID NO: 1)
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEFQERL
QEDTSFGGNLGRAVFQAKKRILEPLGLVEEAAKTAPGKKRPVDQSPQEPDSSSGVGK
SGKQPARKRLNFGQTGDSESVPDPQPLGEPPAAPTSLGSNTMASGGGAPMADNNEG
ADGVGNSSGNWHCDSQWLGDRVITTSTRTWALPTYNNHLYKQISSQSGASNDNHYF
GYSTPWGYFDFNRFHCHFSPRDWQRLINNNWGFRPKKLSFKLFNIQVKEVTQNDGT
TTIANNLTSTVQVFTDSEYQLPYVLGSAHQGCLPPFPADVFMVPQYGYLTLNNGSQA
VGRSSFYCLEYFPSQMLRTGNNFQFSYTFEDVPFHSSYAHSQSLDRLMNPLIDQYLY
YLNRTQGTTSGTTNQSRLLFSQAGPQSMSLQARNWLPGPCYRQQRLSKTANDNNNS
NFPWTAASKYHLNGRDSLVNPGPAMASHKDDEEKFFPMHGNLIFGKEGTTASNAEL
DNVMITDEEEIRTTNPVATEQYGTVANNLQSSNTAPTTRTVNDQGALPGMVWQDRD
VYLQGPIWAKIPHTDGHFHPSPLMGGFGLKHPPPQIMIKNTPVPANPPTTFSPAKFASF
ITQYSTGQVSVEIEWELQKENSKRWNPEIQYTSNYNKSVNVDFTVDTNGVYSEPRPI
GTRYLTRNL

In some embodiments, the modified AAV capsid comprises an amino acid sequence that is at least 80%, at least 85%, at least 90%, at least 95%, at least 98%, or at least 99.5% identical the sequence set forth as SEQ ID NO: 2. In some embodiments, the modified AAV capsid comprises 1, 2, 3, 4, 5, 6, 7, 8, 9, 10, 11, 12, 13, 14, 15, or 15-20 amino acid substitutions relative to the amino acid sequence of SEQ ID NO: 2. In some embodiments, these differences may comprise amino acids that have been inserted, deleted, and/or substituted relative to the sequence of SEQ ID NO: 2. In some embodiments, the disclosed AAV capsid variants comprise truncations at the N- or C-terminus relative to the sequence of SEQ ID NO: 2. In some embodiments, the disclosed AAV capsid variants comprise stretches of 15, 20, 25, 30, 35, 40, 45, 50, or more than 50 consecutive amino acids in common with the sequence of SEQ ID NO: 2.

In some embodiments, the modified or variant AAV capsid comprises the VP1 sequence of AAV3B-V04, or “V04”, which comprises the amino acid sequence set forth as SEQ ID NO: 2:

(SEQ ID NO: 2)
MAADGYLPDWLEDNLSEGIREWWALKPGVPQPKANQQHQDNRRGLVLPGYKYLG
PGNGLDKGEPVNEADAAALEHDKAYDQQLKAGDNPYLKYNHADAEFQERLQEDTS
FGGNLGRAVFQAKKRILEPLGLVEEAAKTAPGKKRPVDQSPQEPDSSSGVGKSGKQP
ARKRLNFGQTGDSESVPDPQPLGEPPAAPTSLGSNTMASGGGAPMADNNEGADGVG
NSSGNWHCDSQWLGDRVITTSTRTWALPTYNNHLYKQISSASGASNDNHYFGYSTP
WGYFDFNRFHCHFSPRDWQRLINNNWGFRPKKLSFKLFNIQVKEVTQNDGTTTIAN
NLTSTVQVFTDSEYQLPYVLGSAHQGCLPPFPADVFMVPQYGYLTLNNGSQAVGRS
SFYCLEYFPSQMLRTGNNFQFSYTFEDVPFHSSYAHSQSLDRLMNPLIDQYLYYLNR
TQSNASGTAGTRGMKFSQAGPQSMSLQARNWLPGPCYRQQRLSKTANDNNNSNFP
WTAASKYHLNGRDSLVNPGPAMASHKDDEEKFFPMHGNLIFGKEGTTASNAELDN
VMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFIT
QYSTGQVSVEIEWELQKENSKRWNPEIQYTSNYNKSVNVDFTVDTNGVYSEPRPIGT
RYLTRNL

In some embodiments, the modified or variant AAV capsid comprises an amino acid sequence that is at least 80%, at least 85%, at least 90%, at least 95%, at least 98%, or at least 99.5% identical the sequence set forth as SEQ ID NO: 10. In some embodiments, the modified or variant AAV capsid comprises 1, 2, 3, 4, 5, 6, 7, 8, 9, 10, 11, 12, 13, 14, 15, or 15-20 amino acid substitutions relative to the amino acid sequence of SEQ ID NO: 10. In some embodiments, these differences may comprise amino acids that have been inserted, deleted, and/or substituted relative to the sequence of SEQ ID NO: 10. In some embodiments, the disclosed AAV capsid variants comprise truncations at the N- or C-terminus relative to the sequence of SEQ ID NO: 10. In some embodiments, the disclosed capsid rAAV variants comprise stretches of 15, 20, 25, 30, 35, 40, 45, 50, or more than 50 consecutive amino acids in common with the sequence of SEQ ID NO: 10.

In some embodiments, the modified or variant AAV capsid comprises the VP1 sequence of AAV3B-V05, or “V05”, which comprises the amino acid sequence set forth as SEQ ID NO: 10:

(SEQ ID NO: 10)
MAADGYLPDWLEDNLSEGIREWWALKPGVPQPKANQQHQDNRRGLVLPGYKYLG
PGNGLDKGEPVNEADAAALEHDKAYDQQLKAGDNPYLKYNHADAEFQERLQEDTS
FGGNLGRAVFQAKKRILEPLGLVEEAAKTAPGKKRPVDQSPQEPDSSSGVGKSGKQP
ARKRLNFGQTGDSESVPDPQPLGEPPAAPTSLGSNTMASGGGAPMADNNEGADGVG
NSSGNWHCDSQWLGDRVITTSTRTWALPTYNNHLYKQISSASGASNDNHYFGYSTP
WGYFDFNRFHCHFSPRDWQRLINNNWGFRPKKLSFKLFNIQVKEVTQNDGTTTIAN
NLTSTVQVFTDSEYQLPYVLGSAHQGCLPPFPADVFMVPQYGYLTLNNGSQAVGRS
SFYCLEYFPSQMLRTGNNFQFSYTFEDVPFHSSYAHSQSLDRLMNPLIDQYLYYLNR
TQSNAGGTANMNRLMFSQAGPQSMSLQARNWLPGPCYRQQRLSTIDTQNNNSNFP
WTAASKYHLNGRDSLVNPGPAMASHKDDEERFFPMHGNLIFGKQGAEASNVEVGK
VMITDEEEIRTTNPVATEQYGTVANNLQSSNTAPTTRTVNDQGALPGMVWQDRDVY
LOGPIWAKIPHTDGHFHPSPLMGGFGLKHPPPQIMIKNTPVPANPPTTFSPAKFASFIT
QYSTGQVSVEIEWELQKENSKRWNPEIQYTSNYNKSVNVDFTVDTNGVYSEPRPIGT
RYLTRNL

Accordingly, in some embodiments, provided herein are AAV3B capsid protein variants comprising substitutions relative to the wild-type AAV3B VP1 sequence (e.g., as set forth in SEQ ID NO: 1). In some embodiments, an amino acid substitution in any one of the AAV3B capsid protein variants disclosed herein lies in a variable region (VR) as defined by wild-type AAV3B VP1 protein. It should be understood that any positioning of an amino acid as described herein is with respect to the sequence of the wild-type AAV3B VP1 sequence as set forth in SEQ ID NO: 1.

In some embodiments, a variant AAV3B capsid comprises one or more amino acid substitutions in at least one variable region (e.g., VRI, VRII, VRIII, VRIV, VRV, VRVI, VRVII, VRVIII or VRIX). In some embodiments, a variant AAV3B capsid comprises one or more amino acid substitutions in more than one variable region (e.g., VRI and VRII, VRI and VRVII or VRIV, VRII).

Certain aspects of the AAV variant capsids and AAV virions of the present disclosure include the second nucleotide encoding variants of an AAV Cap protein as listed in Table 4 (see VRs IV, V, VI, and VII sequences shown in lines 2-86 (numbered top to bottom) and SEQ ID NOs: 9 and 45-357 described therein). Accordingly, in some embodiments, an AAV variant capsid or a capsid of a rAAV particle comprises the sequence of any one of SEQ ID NOs: 9 or 45-357. In some embodiments, an AAV variant capsid or a capsid of a rAAV particle comprises a VR IV which is any one of the VR IV variants shown in Table 4. In some embodiments, an AAV variant capsid or a capsid of a rAAV particle comprises a VR V which is any one of the VR V variants shown in Table 4. In some embodiments, an AAV variant capsid or a capsid of a rAAV particle comprises a VR VI which is any one of the VR VI variants shown in Table 4. In some embodiments, an AAV variant capsid or a capsid of a rAAV particle comprises a VR VII which is any one of the VR VII variants shown in Table 4. In some embodiments, an AAV variant capsid comprise a plurality of the VRs shown in Table 4. For example, in some embodiments, a capsid may comprise a VR IV and a VR VII as shown in Table 4. As a further example, in some embodiments, a capsid may comprise a variant of each one of VRs IV, V, VI, and VII, such as a variant capsid comprising the variant VRs of line 2 in Table 4 which would comprise a VR IV of SEQ ID NO: 45, a VR V of SEQ ID NO: 46, a VR VI of SEQ ID NO: 47, and a VR VII of SEQ ID NO: 48.

In some embodiments, the present disclosure provides novel infectious rAAV virions and viral particles, as well as nucleic acids (e.g., expression constructs) that encode novel AAV virions. In some embodiments, the present disclosure further provides novel nucleic acids encoding one or more selected diagnostic and/or therapeutic agents for delivery to a selected population of mammalian cells, such as human cells, wherein the nucleic acid molecules are comprised within the disclosed rAAV virions and viral particles.

In some embodiments, the present disclosure provides improved rAAV-based expression constructs that encode one or more therapeutic agents useful in the preparation of medicaments. In some embodiments, said medicaments may be used for the prevention, treatment, and/or amelioration of one or more diseases, disorders, or conditions resulting from a deficiency in one or more cellular components. In some embodiments, the present disclosure provides virions comprising modified or variant AAV capsids, as generated after screening of one or more libraries of rAAV-based genetic constructs encoding one or more selected molecules of interest. In some embodiments, the molecule of interest comprises one or more diagnostic and/or therapeutic agents (including, e.g., proteins, polypeptides, peptides, antibodies, antigen binding fragments, siRNAs, RNAis, antisense oligo- and poly-nucleotides, ribozymes, and variants and/or active fragments thereof). In some embodiments, said agents may be used in the diagnosis, prevention, treatment, and/or amelioration of symptoms of mammalian diseases, disorders, conditions, deficiencies, defects, trauma, and/or injury.

In some embodiments, the novel capsids of the infectious virions disclosed herein may have an improved efficiency in transducing one or more of a variety of cells, tissues and organs of interest, when compared to wild-type capsids. In some embodiments, improved AAV capsid variants, and AAV particles (e.g., rAAV particles) comprising said variant capsids, provided herein may transduce one or more selected host cells at higher-efficiencies (and often much higher efficiencies) than AAV particles (e.g., rAAV particles) comprising wild type capsids.

In some embodiments, in the practice of the present disclosure, the transduction efficiency of an rAAV particle comprising a variant capsid will be higher than that of the corresponding wild-type capsid and, for example, will preferably possess a transduction efficiency in a mammalian cell that is at least about 2-fold, at least about 4-fold, at least about 6-fold, at least about 8-fold, at least about 10-fold, at least about 12-fold, or higher than that of a virion that comprises a corresponding, wildtype capsid. In some embodiments, the transduction efficiency of an rAAV particle comprising a variant capsid provided herein will be at least about 15-fold higher, at least about 20-fold higher, at least about 25-fold higher, or at least about 30-fold higher. In some embodiments, the transduction efficiency of an rAAV particle comprising a variant capsid provided herein will be at least about 40-fold, 45-fold, 50-fold, or greater than 50-fold higher than that of a virion that comprises a corresponding, wild-type capsid.

In some embodiments, the rAAV particles (e.g., virions) as described herein may be of different AAV serotypes, and the mutation of one or more of the sequences described herein may result in improved viral vectors, which are capable of higher-efficiency transduction than that of the corresponding, non-substituted vectors (e.g., comprising non-variant capsid sequences, such as vectors comprising capsid sequences that do not comprise substitutions at nucleotide positions encoding VR regions) from which the mutants were prepared. In some embodiments, the virions as described herein may be of an AAV1, AAV2, AAV3, AAV4, AAV5, AAV6, AAV7, AAV8, AAV9, AAV10, AAV11, AAV12, or AAV13 serotype.

In some embodiments, the present disclosure further provides populations and pluralities of the disclosed rAAV particles (e.g., rAAV virions) infectious viral particles, and mammalian host cells that include one or more nucleic acids encoding them. In some embodiments, the disclosed vectors and rAAV particles (e.g., virions) may be comprised within one or more diluents, buffers, physiological solutions or pharmaceutical vehicles, or formulated for administration to a mammal in one or more diagnostic, therapeutic, and/or prophylactic regimens. In some embodiments, the disclosed rAAV particles, rAAV virions, and pluralities thereof, may also be provided in excipient formulations that are acceptable for veterinary administration to selected livestock, exotics, domesticated animals, and companion animals (e.g., pets), as well as to non-human primates, zoological or otherwise captive specimens.

In some embodiments, the mammalian host cells will be human host cells. In some embodiments, a mammalian host cell (e.g., a human host cell) may be, for example, blood cells, stem cells, bone marrow cells (e.g., hematopoietic cells), CD34 cells, liver or hepatic cells, cancer cells, vascular cells, pancreatic cells, neural cells, ocular or retinal cells, epithelial or endothelial cells, dendritic cells, fibroblasts, lung cells, cardiac cells, intestinal cells, diaphragmatic cells, renal (i.e., kidney) cells, neural cells, or a cell from found in any tissue of a mammal (e.g., a human) or a mammalian cell of a cell line for which viral-based gene therapy is contemplated.

In some embodiments, the present disclosure further provides compositions and formulations that include one or more of the host cells or viral particles of the present disclosure together with one or more pharmaceutically acceptable buffers, diluents, or carriers. In some embodiments, such compositions may be included in one or more diagnostic or therapeutic kits, for diagnosing, preventing, treating or ameliorating one or more symptoms of a mammalian disease, injury, disorder, trauma, or condition.

In some embodiments, the present disclosure further includes methods for providing a mammal in need thereof with a diagnostically- or therapeutically-effective amount of a selected biological molecule. In some embodiments, the method comprises providing to a cell, tissue, and/or organ of a mammal in need thereof, an amount of an rAAV particle (e.g., an rAAV particle comprising a transgene encoded by an expression construct); and for a time effective to provide the mammal with a diagnostically- or a therapeutically-effective amount of the selected biological molecule.

In some embodiments, the present disclosure further provides methods for diagnosing, preventing, treating, or ameliorating at least one or more symptoms of a disease, a disorder, a condition, an injury, an abnormal condition, or trauma in a mammal. In some embodiments, in an overall and general sense, the methods include at least the step of administering to a mammal in need thereof one or more of the disclosed rAAV particles (e.g., an rAAV particle comprising a heterologous nucleic acid, such as an expression construct or transgene), in an amount and for a time sufficient to diagnose, prevent, treat or ameliorate the one or more symptoms of the disease, disorder, condition, injury, abnormal condition, or trauma in the mammal.

In some embodiments, the present disclosure also provides methods of transducing at least one or more (e.g., a population) of mammalian cells. In some embodiments, in an overall and general sense, the methods include at least the step of introducing into one or more cells (e.g., cells of a population), a composition that comprises an effective amount of one or more of the rAAV particles (e.g., rAAV virions) disclosed herein.

In some embodiments, the present disclosure provides compositions, as well as therapeutic and/or diagnostic kits that include one or more of the disclosed AAV compositions (e.g., compositions comprising rAAV particles), formulated with one or more additional ingredients, or prepared with one or more instructions for their use.

In some embodiments, the present disclosure provides methods for using the disclosed improved rAAV particles (e.g., virions) in a variety of ways, including, for example, ex situ, ex vivo, in vitro and in vivo applications, methodologies, diagnostic procedures, and/or gene therapy regimens. In some embodiments, because many of the improved vectors described herein are also resistant to proteasomal degradation, they possess significantly increased transduction efficiencies in vivo making them particularly well suited for viral particle-based human gene therapy regimens, and in particular, for delivering one or more genetic constructs to selected mammalian cells in vivo and/or in vitro.

In some embodiments, the present disclosure provides compositions comprising AAV particles (e.g., rAAV virions and rAAV particles), and pharmaceutical formulations thereof, useful in methods for delivering genetic material encoding one or more beneficial or therapeutic product(s) to mammalian cells and tissues. In some embodiments, the compositions and methods of the present disclosure provide a significant advancement in the art through their use in the treatment, prevention, and/or amelioration of symptoms of one or more mammalian diseases. In some embodiments, it is contemplated that human gene therapy will particularly benefit from the present teachings by providing new and improved viral vector constructs for use in the treatment of a number of diverse diseases, disorders, and conditions.

Contemplated herein are also variant AAV capsid proteins of serotypes other than serotype 3B. In some embodiments, the amino acid substitutions described herein are in a variable region of the AAV capsid protein of a serotype other than serotype 3B that is homologous to the variable region of AAV3B. In some embodiments, a variant AAV capsid protein of a serotype other than serotype 3B is of any serotype other than AAV3B (e.g., AAV serotype 1, 2, 3, 4, 5, 6, 7, 8, 9, 10, 11, 12 or 13). In some embodiments, a variant AAV capsid protein of a serotype other than serotype 3B is of a closely related serotype (e.g., AAV3).

### Library Design And Construction

In some embodiments, the present disclosure concerns libraries of AAV capsid variants that demonstrate improved properties useful in the delivery of one or more therapeutic agents to selected mammalian cells, and particularly for use in the prevention, treatment, and/or amelioration of one or more disorders in a mammal into which the vector construct may be introduced. In some embodiments, the disclosed libraries comprise AAV3 capsid variants.

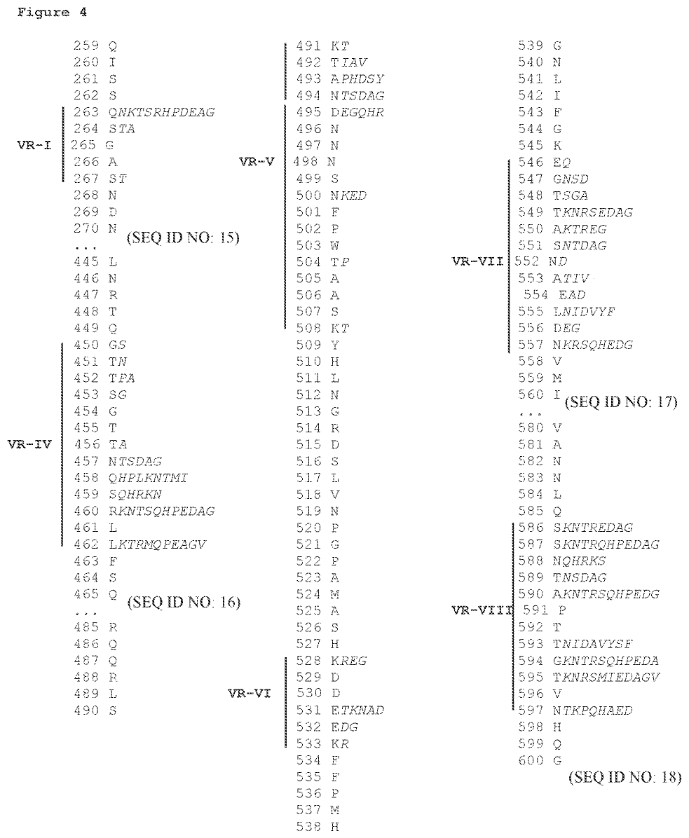

Comparison of the AAV VP3 structure among various serotypes has revealed highly homologous sequences interspersed with more evolutionary divergent areas. These amino acid stretches are commonly designated as VRs I through IX (variable regions I-IX; also known as “loops”). VRs are localized at the surface of the assembled capsid and are assumed to be responsible for the capsid interaction with cell surface receptors and other host factors. Because of their location, VRs are also predicted to be less critical for capsid assembly. Therefore, the guiding principle of the library's design was to modify only surface VRs while keeping the backbone sequence unchanged to maintain the integrity of the assembling scaffold. All candidate positions for mutagenesis in the AAV3 background, were selected from the alignment of known variants, which can be evaluated on a three-dimensional model of the AAV3 capsid. The amino acid diversity of VR-I, VR-IV, VR-V, VR-VI, VR-VII and VR-VIII is shown in FIG. 4. AAV3 wildtype VR-II, VR-III and VR-IX and non-variable regions of VP3 were incorporated in the plasmid library. The wild-type AAV3B sequence is set forth in SEQ ID NO: 1.

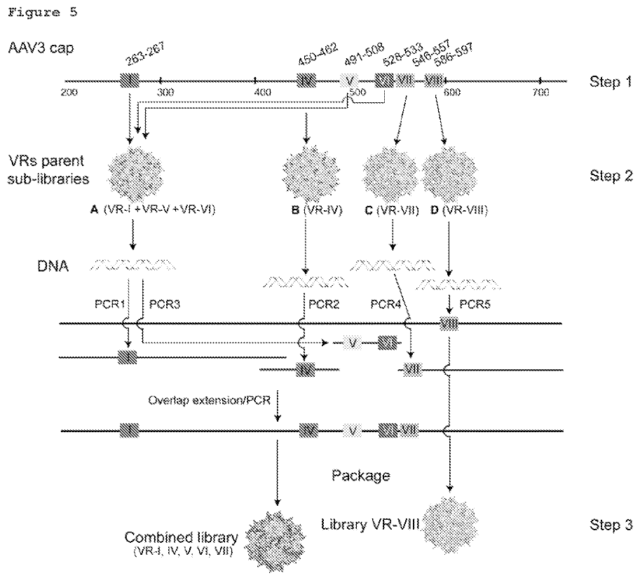

The AAV3B library of the present disclosure was built in three steps: first, VR parent sub-libraries were prepared each containing mutations in only one VR (B: VR-IV, C: VR-VII, D: VR-VIII) or a subset of VRs (A: VR-I+VR-V+VR-VI), then, structurally compatible sequences were combined to generate master libraries (A+B+C: VRs I, IV, V, VI, VII) and (D: VR-VIII), and finally the master libraries were packaged. See Example 1 and FIG. 5. Methods for generating and assembling DNA fragments for the library are disclosed in International Publication Nos. WO 2015/048534 and WO 2017/070476, and U.S. Pat. No. 7,220,577, each of which are incorporated herein by reference. The completed master library comprised 10⁷ variants.

The amino acid substitutions in the wild-type AAV3B capsid proteins disclosed herein are epistatic, i.e. that they interact with one another, e.g. synergistically. The disclosed substitutions may be grouped into motifs of substitutions. In designing the disclosed library, motifs were introduced to the capsids simultaneously and stochastically, rather than once at a time. The substitutions in each capsid variant were determined to be epistatic and act synergistically on capsid binding and transduction behavior. These motifs confer unexpectedly enhanced transduction efficiencies and may confer an ability to evade neutralizing antibodies relative to wild-type capsids of the prior art.

### Tissue-Specific Or Cell-Specific Virions

In some embodiments, the master library may be used to select virions having capsids containing degenerate or otherwise variant Cap protein (i.e., Cap protein that differs from wildtype serotype 3 at least at one amino acid position) that are targeted to particular tissue or cell types. For example, in some embodiments, virions made according to the present disclosure include those that exhibit a new tropism, e.g., those capable of infecting cells normally non-permissive to AAV infection in general or at least non-permissive to AAV3 infection, as well as those that exhibit an increased or decreased ability to infect a particular cell or tissue type. As another example, in some embodiments, virions made according to the present disclosure include those that lack the ability to infect cells normally permissive to AAV infection in general or at least normally permissive to AAV3 infection. In some embodiments, to select for virions having a particular cell- or tissue-specific tropism, a packaged master library is introduced into a target cell. In some embodiments, the target cell is also infected with a helper virus (e.g. adenovirus, or Ad). The target cell is cultured under conditions that allow for the production of virions, resulting in a population of virions that are harvested from the target cell. This population of virions has been selected for having a tropism for that target cell.

In some embodiments, as controls in a typical experiment in which virions having a particular tropism are selected, cells in different flasks or dishes may be simultaneously infected with WT AAV3 or rAAV particles using the same conditions as used for the library. In some embodiments, after a suitable time post-infection, cells may be harvested, washed and the virions purified using a suitable purification method. See Gao et al., Hum. Gene Ther. 9:2353-62, 1998; U.S. Pat. No. 6,146,874; and Zolotukhin et al., Gene Ther. 6:973-85, 1999, each of which are incorporated herein by reference. In some embodiments, AAV and helper virions (e.g., Ad) from each infection may be titered, e.g. by real-time PCR, and the AAV virions may then be further propagated, resulting in a stock of selected virions.

In some embodiments, once the selected population of virions having a desired tropism is isolated, nucleic acid from the virions is isolated and the sequence of the nucleotide sequence encoding the at least one AAV Cap protein is determined. In some embodiments, virions constructed and selected according to the present disclosure (e.g. virions comprising V04 and V05) are useful for specifically targeting diseased cells, or tissues thereof, over non-diseased cells, or tissues thereof.

Alternatively, in some embodiments, tissue- or cell-specific virions may be selected using an in vivo approach. For example, in some embodiments, mice (or other suitable host) may be injected with a suitable amount of viral preparation (e.g., 1×10¹⁰ to 1×10¹¹ vector genomes (vg) in the case of mice) via the tail vein. In some embodiments, as described in Example 2, more than one round of selection (iterative selection) may be performed by injecting the original master library for the first round and target-enriched libraries in subsequent rounds. In some embodiments, hosts are euthanized after an incubation period (3 to 4 days for mice), and episomal DNA is purified from the target cells or tissue and used as a template to amplify capsid DNA sequences. In some embodiments, target-enriched libraries may then be generated, purified, and quantified. In some embodiments, after several rounds of selection, amplified capsid DNA may be inserted into an appropriate vector for cloning and random clones may be analyzed by sequencing.

### Expression Constructs

In some embodiments, the present disclosure provides polynucleotide expression constructs that encode one or more of the disclosed capsids as described herein. In some embodiments, the expression construct may be comprised within a plasmid. These plasmids may comprise one or more nucleotide substitutions in the nucleic acid sequence that encodes a wild-type AAV3B capsid, e.g., one or more nucleotide substitutions in one or more variable regions.

In some embodiments, the nucleic acid vector comprises one or more transgenes comprising a sequence encoding a protein or polypeptide of interest operably linked to a promoter, wherein the nucleic acid comprising the one or more transgenes is flanked on each side with an ITR sequence. In some embodiments, the ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV3. In some embodiments, the ITR sequences of the first serotype are derived from AAV1, AAV5, AAV6, AAV7, AAV8, AAV9 or AAV10. In some embodiments, the ITR sequences are of the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the present disclosure provides rAAV nucleic acid vectors that may comprise a nucleic acid segment comprising a promoter, an enhancer, a post-transcriptional regulatory sequence, a polyadenylation signal, or any combination thereof. In some embodiments, said regulatory elements are operably linked to a nucleic acid segment that encodes the selected polynucleotide of interest (e.g., a transgene). In some embodiments, the promoter is a heterologous promoter, a tissue-specific promoter, a cell-specific promoter, a constitutive promoter, an inducible promoter, or any combination thereof. In some embodiments, the expression constructs of the present disclosure further include at least promoter capable of expressing, or directed to primarily express, the nucleic acid segment in a suitable host cell (e.g., a liver cell) comprising the vector.

In some embodiments, nucleic acids cloned into one or more of the novel rAAV nucleic acid vectors described herein will express or encode one or more transgenes of interest. In some embodiments, transgenes of interest may comprise polypeptides, peptides, ribozymes, peptide nucleic acids, siRNAs, RNAis, antisense oligonucleotides, antisense polynucleotides, antibodies, antigen binding fragments, growth factors, cytokines and other small therapeutic proteins, or any combination thereof. In some embodiments, the one or more transgenes encode an antibody, secreted growth factor, or cytokine.

In some embodiments, the transgene of interest encodes a serine protease inhibitor. In some embodiments, the transgene comprises the SERPINA1 gene (e.g., the human SERPINA1 gene), which encodes alpha-1-antitrypsin in humans (UniProtKB accession number: P01009). In some embodiments, the transgene encodes a transport protein. In certain embodiments, the transgene comprises the TTR gene (e.g., the human TTR gene), which encodes transthyretin in humans (UniProtKB accession number: P02766). In some embodiments, the transgene encodes a P-type ATPase. In certain embodiments, the transgene comprises the ATP7B gene (e.g., the human ATP7B gene), which encodes a copper-transporting P-type ATPase in humans (UniProtKB accession number: P35670). In some embodiments, the transgene encodes a carbamoyltransferase. In certain embodiments, the transgene comprises the OTC gene (e.g., the human OTC gene), which encodes ornithine transcarbamylase in humans (UniProtKB accession number: P00480).

In some embodiments, therapeutic agents useful in the disclosed vectors may include one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

In some embodiments, the rAAV nucleic acid vectors obtained by the disclosed methods may encode at least one diagnostic and/or therapeutic protein or polypeptide selected from the group consisting of a molecular marker, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, a tumor suppressor, and any combination thereof.

In some embodiments, the rAAV nucleic acid vectors of the present disclosure may comprise one or more nucleic acid segments that encode a polypeptide selected from the group consisting of BDNF, CNTF, CSF, EGF, FGF, G-SCF, GM-CSF, gonadotropin, IFN, IFG-1, M-CSF, NGF, PDGF, PEDF, TGF, TGF-B2, TNF, VEGF, prolactin, somatotropin, XIAP1, IL-1, IL-2, IL-3, IL-4, IL-5, IL-6, IL-7, IL-8, IL-9, IL-10, IL-10(I87A), viral IL-10, IL-11, IL-12, IL-13, IL-14, IL-15, IL-16, IL-17, IL-18, and any combination thereof.

In some embodiments, the rAAV nucleic acid vectors of the present disclosure may further include one or more enhancer sequences. In some embodiments, one or more enhancers are each operably linked to a nucleic acid of interest (e.g., one encoding a transgene). Non-limiting examples of enhancer sequences include a CMV enhancer, a synthetic enhancer, a liver-specific enhancer, a vascular-specific enhancer, a brain-specific enhancer, a neural cell-specific enhancer, a lung-specific enhancer, a muscle-specific enhancer, a kidney-specific enhancer, a pancreas-specific enhancer, retinal-specific enhancer, and an islet cell-specific enhancer.

Non-limiting examples of promoters useful in the practice of the present disclosure includeone or more heterologous, tissue-specific, constitutive or inducible promoters, such as a promoter selected from the group consisting of a CMV promoter, a β-actin promoter, an insulin promoter, an enolase promoter, a BDNF promoter, an NGF promoter, an EGF promoter, a growth factor promoter, an axon-specific promoter, a dendrite-specific promoter, a brain-specific promoter, a hippocampal-specific promoter, a kidney-specific promoter, a retinal-specific promoter, an elafin promoter, a cytokine promoter, an interferon promoter, a growth factor promoter, an α₁-antitrypsin promoter, a brain cell-specific promoter, a neural cell-specific promoter, a central nervous system cell-specific promoter, a peripheral nervous system cell-specific promoter, an interleukin promoter, a serpin promoter, a hybrid CMV promoter, a hybrid β-actin promoter, an EF1 promoter, a U1a promoter, a U1b promoter, a Tet-inducible promoter, a VP1 6-LexA promoter, or any combination thereof. In some embodiments, the promoter may include a mammalian or avian β-actin promoter.

In some embodiments, the vector-encoding nucleic acid segments further comprise one or more post-transcriptional regulatory sequences or one or more polyadenylation signals, such as a woodchuck hepatitis virus post-transcription regulatory element (WPRE), a polyadenylation signal sequence, or any combination thereof.

In some embodiments, the present disclosure provides rAAV nucleic acid vectors (e.g., genetically modified vectors) with improved transduction efficiency that include at least a first nucleic acid segment that encodes one or more therapeutic agents that alter, inhibit, reduce, prevent, eliminate, or impair the activity of one or more endogenous biological processes in the cell. In some embodiments, such therapeutic agents may be those that selectively inhibit or reduce the effects of one or more metabolic processes, conditions, disorders, or diseases. In some embodiments, the defect may be caused by injury or trauma to the mammal for which treatment is desired. In some embodiments, the defect may be caused the over-expression of an endogenous biological compound. In some embodiments, the defect may be caused by the under-expression or even lack of one or more endogenous biological compounds.

In some embodiments, the rAAV nucleic acid vectors of the present disclosure may also further include a second distinct nucleic acid segment that comprises, consists essentially of, or consists of, one or more enhancers, one or more regulatory elements, one or more transcriptional elements, or any combination thereof, that alter, improve, regulate, and/or affect the transcription of the nucleotide sequence of interest expressed by the rAAV vectors.

For example, in some embodiments, the rAAV nucleic acid vectors of the present disclosure may further include a second nucleic acid segment that comprises, consists essentially of, or consists of, a CMV enhancer, a synthetic enhancer, a cell-specific enhancer, a tissue-specific enhancer, or any combination thereof. In some embodiments, the second nucleic acid segment may also further comprise, consist essentially of, or consist of, one or more intron sequences, one or more post-transcriptional regulatory elements, or any combination thereof.

In some embodiments, the vectors of the present disclosure may also optionally further include a polynucleotide that comprises, consists essentially of, or consists of, one or more polylinkers, restriction sites, and/or multiple cloning region(s). In some embodiments, said sequences facilitate insertion (cloning) of one or more selected genetic elements, genes of interest, or therapeutic or diagnostic constructs into the rAAV construct at a selected site within the construct.

In some embodiments, the disclosed nucleic acid vectors may be self-complementary (i.e., scrAAV nucleic acid vectors). In some embodiments, the vectors may be single-stranded.

In some embodiments, expression constructs and nucleic acid vectors of the present disclosure may be prepared in a variety of compositions and may also be formulated with appropriate pharmaceutical vehicles for administration to human or animal subjects.

### Host Cells

In some embodiments, the present disclosure also provides host cells that comprise at least one or more of the nucleic acids disclosed herein. In some embodiments, a host cell comprises a nucleic acid comprising a sequence encoding an AAV variant capsid protein disclosed herein (e.g., see SEQ ID NOs: 9 or 45-357). In some embodiments, a host cell (e.g., a cell in a tissue of subject, such as a diseased tissue in a subject with a disease or disorder) comprises a nucleic acid which has been delivered to the cell by an rAAV particle comprising a variant capsid. In some embodiments, a host cell (e.g., a packaging host cell for producing viral particles) comprises an AAV or rAAV particle comprising a nucleic acid disclosed herein.

In some embodiments, a host cell comprises a nucleic acid comprising V04 and V05). In some embodiments, a host cell comprises or one or more of the disclosed rAAV expression constructs. In some embodiments, host cells are mammalian host cells. In some embodiments, a host cell is a human host cell. In some embodiments, a host cell may be an isolated (e.g., isolated from a subject) and/or in cell or tissue culture. In some embodiments, a host cell is located in the body (e.g., in liver tissue) of an animal model (e.g., a genetically modified animal model). In some embodiments, the host cells comprise humanized host cells. In some embodiments, the host cells comprise humanized hepatocytes.

Examples of suitable host cells include hepatocytes, such as H2.35, HUH-7 and HepG2, HEK293 embryonic kidney cells, HeLa cells, Cos cells, U87 cells, KB cells, and Vero cells. In some embodiments, nucleic acids of the modified or variant AAV3B virions of the present disclosure are incorporated into HUH-7 and/or HepG2 cells. In some embodiments, virions comprising the V04 and/or V05 variants are incorporated into HUH-7 and/or HepG2 cells.

As described above, in some embodiments, the exogenous polynucleotide will encode one or more proteins, polypeptides, peptides, ribozymes, or antisense oligonucleotides, or a combination of these. In some embodiments, the exogenous polynucleotide may encode two or more such molecules, or a plurality of such. In some embodiments, when combinational gene therapies are desired, two or more different molecules may be produced from a single rAAV expression construct, or alternatively, a selected host cell may be transfected with two or more unique rAAV expression constructs, each of which will provide unique transgenes encoding at least two different such molecules.

Use Of rAAV Virions In Prophylaxis, Diagnosis, Or Therapy

In some embodiments, the present disclosure also provides for uses of the compositions disclosed herein as a medicament, or in the manufacture of a medicament, for treating, preventing or ameliorating the symptoms of a disease, disorder, condition, injury or trauma, including, but not limited to, the treatment, prevention, and/or prophylaxis of a disease, disorder or condition, and/or the amelioration of one or more symptoms of such a disease, disorder or condition.

In some embodiments, the disease, disorder or condition consists of Alpha-1 Antitrypsin Deficiency, Transthyretin-Related Familial Amyloid Polyneuropathy, Ornithine Transcarbamylase Deficiency, Fabry Disease, Pompe Disease, Galactosemia, Progressive Familial Intrahepatic Cholestasis Types 1, 2 and 3, Hereditary Angioedema, Hemophilia B, Hemophilia A, Phenylketonuria, Glycogen Storage Disease Type 1A, Wilson's Disease, or Citrullinemia.

In some embodiments, the disease, disorder or condition is Alpha-1 Antitrypsin Deficiency, a rare inherited condition that results from deficiency of alpha-1 antitrypsin (AAT), a protein produced in the liver encoded by the SERPINA1 gene. AAT deficiency often leads to cirrhosis and other severe liver diseases, as well as emphysema and COPD in the lungs.

In some embodiments, the disease, disorder or condition is Transthyretin-Related Familial Amyloid Polyneuropathy (FAP), or Familial Transthyretin Amyloidosis (FTA). FAP is a rare inherited condition that results from an abnormal accumulation of amyloid in the body's tissues, and in particular liver tissue, due to abnormal misfolding and aggregation of transthyretin. FAP is an autosomal dominant condition resulting from a mutation in the TTR gene. In the absence of a liver transplant, FAP is invariably fatal.

In some embodiments, the disease, disorder or condition is Wilson's Disease. Wilson's disease is a rare inherited disorder that causes copper to accumulate in organs such as the liver. It is an autosomal recessive condition due to a mutation in the ATP7B gene, which encodes a P-type ATPase that transports copper into bile and incorporates it into ceruloplasmin.

In some embodiments, the disease, disorder or condition is Ornithine Transcarbamylase Deficiency (OTC Deficiency), an inherited condition that results from a toxic accumulation of ammonia in the blood. It is an X-linked recessive condition due to a mutation in the OTC gene, which encodes a carbamoyltransferase that is expressed only in the liver and is responsible for converting carbamoyl phosphate and ornithine into citrulline as part of the urea cycle.

In some embodiments, the creation of recombinant non-human host cells, humanized host cells, and/or isolated recombinant human host cells that comprise one or more nucleic acids of the disclosed rAAV virions (e.g., virions comprising V04 and/or V05) is also contemplated to be useful for a variety of diagnostic, and laboratory protocols, including, for example, means for the production of large-scale quantities of the virions described herein. In some embodiments, such virus production methods may comprise improvements over existing methodologies including in particular those that require very high titers of the viral stocks in order to be useful as a gene therapy tool. The inventors contemplate that, in some embodiments, one very significant advantage of the present methods will be the ability to utilize lower titers of viral particles in mammalian transduction protocols, yet still retain transfection rates at a suitable level.

In some embodiments, the present disclosure provides methods of transducing a hepatic cell with a transgene of interest, the method comprising providing to the hepatic cell any of the recombinant AAV particles comprising a variant capsid of the disclosure. In some embodiments, the hepatic cell is a human hepatocyte.

In some embodiments, the present disclosure provides methods of use of the disclosed virions, expression constructs, compositions, and host cells in the preparation of medicaments for diagnosing, preventing, treating or ameliorating at least one or more symptoms of a disease, a condition, a disorder, an abnormal condition, a deficiency, injury, or trauma in an animal, and in particular, in a vertebrate mammal, e.g., Alpha-1 Antitrypsin Deficiency, Transthyretin-Related Familial Amyloid Polyneuropathy, Ornithine Transcarbamylase Deficiency, or Wilson's Disease. In some embodiments, such methods generally involve administration to a mammal in need thereof, one or more of the disclosed virions, host cells, compositions, or pluralities thereof, in an amount and for a time sufficient to diagnose, prevent, treat, or lessen one or more symptoms of such a disease, condition, disorder, abnormal condition, deficiency, injury, or trauma in the affected animal. In some embodiments, the methods may also encompass prophylactic treatment of animals suspected of having such conditions, or administration of such compositions to those animals at risk for developing such conditions either following diagnosis, or prior to the onset of symptoms.

In some embodiments, the present disclosure also provides a method for treating or ameliorating the symptoms of such a disease, injury, disorder, or condition in a mammal. In some embodiments, such methods generally involve at least the step of administering to a mammal in need thereof, one or more of the rAAV virions as disclosed herein, in an amount and for a time sufficient to treat or ameliorate the symptoms of such a disease, injury, disorder, or condition in the mammal. In some embodiments, such treatment regimens are particularly contemplated in human therapy, via administration of one or more compositions either intramuscularly, intravenously, subcutaneously, intrathecally, intraperitoneally, or by direct injection into an organ or a tissue of the mammal under care.

In some embodiments, the present disclosure also provides a method for providing to a mammal in need thereof, a therapeutically-effective amount of an rAAV composition of the present disclosure, in an amount, and for a time effective to provide the patient with a therapeutically-effective amount of the desired therapeutic agent(s) encoded by one or more nucleic acid segments comprised within the rAAV virion, e.g. a virion comprising V04 and/or V05. Non-limiting examples of therapeutic agents include a polypeptide, a peptide, an antibody, an antigen-binding fragment, a cytokine, a ribozyme, a peptide nucleic acid, an siRNA, an RNAi, an antisense oligonucleotide, an antisense polynucleotide, or a combination thereof.

In some embodiments, because the rAAV capsid variants of the disclosure possess enhanced ability to reduce seroreactivity and evade neutralizing antibodies, the compositions and methods provided herein facilitate the re-dosing or re-administration of an rAAV particle comprising any of the disclosed capsid variants to a subject who has been administered an rAAV particle previously, e.g., as part of a therapeutic regimen. In some embodiments, this reduced seroreactivity likewise facilitates the first administration of an rAAV particle to a subject who had exposure to rAAVs previously naively, or outside of the context of a therapeutic regimen. In some embodiments, these subjects are human.

Accordingly, in some embodiments, the present disclosure provides re-dosing regimens of rAAV. In some embodiments of the disclosure, methods of re-administration of rAAV particles (or virions) are provided. In some embodiments, such methods may comprise a first administration, followed by a subsequent (or second) administration of an rAAV particle comprising any of the disclosed capsid variants. In some embodiments, such methods comprise re-administering the recombinant AAV particle or a composition comprising such a particle to the subject, e.g., a human subject in need thereof who has previously been administered the recombinant AAV particle or the composition.

### Pharmaceutical Compositions and Kits

In some embodiments, the present disclosure provides compositions comprising one or more of the disclosed rAAV particles or rAAV virions (e.g. virions comprising V04 and/or V05), expression constructs, infectious AAV particles, or host cells. In some embodiments, provided herein are compositions of rAAV particles or rAAV virions that further comprise a pharmaceutically acceptable carrier for use in therapy, and for use in the manufacture of medicaments for the treatment of one or more mammalian diseases, disorders, conditions, or trauma (e.g., AAT or FAP). In some embodiments, such pharmaceutical compositions may optionally further comprise one or more diluents, buffers, liposomes, a lipid, a lipid complex, a microsphere or a nanoparticle.

In some embodiments, the disclosure provides pharmaceutical compositions that comprise a modified rAAV vector as disclosed herein, and further comprise a pharmaceutical excipient, and may be formulated for administration to host cell ex vivo or in situ in an animal, such as a human. In some embodiments, such compositions comprise a liposome, a lipid, a lipid complex, a microsphere, a microparticle, a nanosphere, or a nanoparticle, or may be otherwise formulated for administration to the cells, tissues, organs, or body of a subject in need thereof. In some embodiments, such compositions are formulated for use in a variety of therapies, such as in the amelioration, prevention, and/or treatment of conditions, such as peptide deficiency, polypeptide deficiency, peptide overexpression, polypeptide overexpression, including for example, conditions, diseases, or disorders as described herein.

In some embodiments, the number of rAAV particles administered to a subject may be on the order ranging from 10⁶ to 10¹⁴ particles or 10³ to 10¹³ particles, or any values therebetween for either range, such as for example, about 10⁶, 10⁷, 10⁸, 10⁹, 10¹⁰, 10¹¹, 10¹², 10¹³, or 10¹⁴ particles. In some embodiments, rAAV particles of higher than 10¹³ particles are be administered. In some embodiments, the number of rAAV particles administered to a subject may be on the order ranging from 10⁶ to 10¹⁴ vector genomes(vgs) or 10³ to 10¹⁵ vgs, or any values there between for either range, such as for example, about 10⁶, 10⁷, 10⁸, 10⁹, 10¹⁰, 10¹¹, 10¹², 10¹³, or 10¹⁴ vgs. In some embodiments, a dose of between 1×10¹² and 4×10¹²vgs (or between 5×10¹¹ to 2×10¹² vgs/kg of the subject) is administered to the subject. In some embodiments, any of the above-described doses of particles or viral genomes may be suspended in a liquid composition (e.g., a composition comprising 10³ to 10¹⁴ particles or vector genomes/mL). In some embodiments, said composition may be administered to a subject in a volume of 0.01-10.0 mL.

In some embodiments, the rAAV particles can be administered as a single dose, or divided into two or more administrations as may be required to achieve therapy of the particular disease or disorder being treated.

In some embodiments, where a second nucleic acid vector encoding the Rep protein within a second rAAV particle is administered to a subject, the ratio of the first rAAV particle to the second rAAV particle is 1:100, 1:50, 1:40, 1:30, 1:20, 1:10, 1:5, 1:2 or 1:1. In some embodiments, the Rep protein is delivered to a subject such that target cells within the subject receive at least two Rep proteins per cell.

In some embodiments, the disclosure provides formulations of compositions disclosed herein in pharmaceutically acceptable carriers for administration to a cell or an animal, either alone or in combination with one or more other modalities of therapy, and in particular, for therapy of human cells, tissues, and diseases affecting man.

In some embodiments, if desired, rAAV particles or preparations, Rep proteins, and/or nucleic acid vectors may be administered in combination with other agents as well, such as proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. In some embodiments, the rAAV particles or preparations, Rep proteins, and/or nucleic acid vectors may thus be delivered along with various other agents as required in the particular instance. In some embodiments, such compositions may be purified from host cells or other biological sources, or alternatively may be chemically synthesized as described herein.

The formulation of pharmaceutically acceptable carriers is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

In some embodiments, these formulations may contain at least about 0.1% of the therapeutic agent (e.g., rAAV particle or preparation, Rep protein, and/or nucleic acid vector) or more, although the percentage of the active ingredient(s) may, of course, be varied and may conveniently be between about 1% or 2% and about 70% or 80% or more of the weight or volume of the total formulation. In some embodiments, the amount of therapeutic agent(s) in each therapeutically useful composition may be prepared is such a way that a suitable dosage will be obtained in any given unit dose of the compound. In some embodiments, factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, delivery of the rAAV particles or preparations, Rep proteins, and/or nucleic acid vectors in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

In some embodiments, the pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. In some embodiments, the carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. In some embodiments, proper fluidity may be maintained, for example, by the use of a coating, such as lecithin, by the maintenance of the required particle size in the case of dispersion and by the use of surfactants.

In some embodiments, the pharmaceutical compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

In some embodiments, for administration of an injectable aqueous solution, for example, the solution may be suitably buffered, if necessary, and the liquid diluent first rendered isotonic with sufficient saline or glucose. In some embodiments, these particular aqueous solutions are especially suitable for intravenous, intramuscular, intravitreal, subcutaneous and intraperitoneal administration. To this end, in some embodiments, a sterile aqueous medium that can be employed will be known to those of skill in the art in light of the present disclosure. For example, in some embodiments, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Ed., 1035-1038 and 1570-1580). In some embodiments, some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

In some embodiments, sterile injectable solutions are prepared by incorporating the rAAV particles or preparations, Rep proteins, and/or nucleic acid vectors, in the required amount in the appropriate solvent with several of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders for the preparation of sterile injectable solutions are prepared by vacuum-drying and freeze-drying techniques which yield a powder of the active ingredient plus any additional desired ingredient from a previously sterile-filtered solution thereof.

Ex vivo delivery of cells transduced with rAAV particles or preparations, and/or Rep proteins is also contemplated herein. In some embodiments, ex vivo gene delivery may be used to transplant rAAV-transduced host cells back into the host. A suitable ex vivo protocol may include several steps. For example, in some embodiments, a segment of target tissue or an aliquot of target fluid may be harvested from the host and rAAV particles or preparations, and/or Rep proteins may be used to transduce a nucleic acid vector into the host cells in the tissue or fluid. In some embodiments, these genetically modified cells may then be transplanted back into the host. In some embodiments, several approaches may be used for the reintroduction of cells into the host, including intravenous injection, intraperitoneal injection, or in situ injection into target tissue. In some embodiments, autologous and allogeneic cell transplantation may be used according to the invention.

In some embodiments, the amount of rAAV particle or preparation, Rep protein, or nucleic acid vector compositions and time of administration of such compositions will be within the purview of the skilled artisan having benefit of the present teachings. In some embodiments, administration of therapeutically-effective amounts of the disclosed compositions may be achieved by a single administration, such as for example, a single injection of sufficient numbers of infectious particles to provide therapeutic benefit to the patient undergoing such treatment. Alternatively, in some embodiments, it may be desirable to provide multiple, or successive administrations of the rAAV particle or preparation, Rep protein, or nucleic acid vector compositions, either over a relatively short, or a relatively prolonged period of time, as may be determined by the medical practitioner overseeing the administration of such compositions.

In some embodiments, toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy of the therapeutic index can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED₅₀ with little or no toxicity. In some embodiments, the dosage may vary within this range depending upon the dosage form employed and the route of administration utilized.

In some embodiments, the present disclosure provides compositions including one or more of the disclosed rAAV particles or rAAV virions (e.g., virions comprising V04 and/or V05) comprised within a kit for diagnosing, preventing, treating or ameliorating one or more symptoms of a mammalian disease, injury, disorder, trauma or condition. In some embodiments, the disease or disorder is AAT or FAP. In some embodiments, such kits may also be useful in the diagnosis, prophylaxis, and/or therapy or a human disease, and may be particularly useful in the treatment, prevention, and/or amelioration of one or more symptoms of Alpha-1 Antitrypsin Deficiency, Transthyretin-Related Familial Amyloid Polyneuropathy, Ornithine Transcarbamylase Deficiency, Wilson's Disease, wet age-related macular degeneration, dry age-related macular degeneration, glaucoma, retinitis pigmentosa, diabetic retinopathy, orphan ophthalmological diseases, cancer, diabetes, autoimmune disease, kidney disease, cardiovascular disease, pancreatic disease, intestinal disease, liver disease, neurological disease, neuromuscular disorder, neuromotor deficit, neuroskeletal impairment, neurological disability, neurosensory condition, stroke, ischemia, Batten's disease, Alzheimer's disease, sickle cell disease, 0-thalassemia, Huntington's disease, Parkinson's disease, skeletal disease, trauma, or pulmonary disease in a human.

In some embodiments, kits comprising one or more of the disclosed rAAV particles or rAAV virions, transformed host cells or pharmaceutical compositions comprising such vectors; and instructions for using such kits in one or more therapeutic, diagnostic, and/or prophylactic clinical embodiments are also provided in the present disclosure. In some embodiments, such kits may further comprise one or more reagents, restriction enzymes, peptides, therapeutics, pharmaceutical compounds, or means for delivery of the composition(s) to host cells, or to an animal (e.g., syringes, injectables, and the like). Exemplary kits include those for treating, preventing, or ameliorating the symptoms of a disease, deficiency, condition, and/or injury, or may include components for the large-scale production of the viral vectors themselves, such as for commercial sale, or for use by others, including e.g., virologists, medical professionals, and the like.

Methods of Making rAAV3B Particles

Various methods of producing rAAV particles (e.g. particles comprising V04 and/or V05) and nucleic acid vectors are known (see, e.g., Zolotukhin et al. Methods 28 (2002) 158-167; and U.S. Patent Publication Nos. US 2007/0015238 and US 2012/0322861, each of which are incorporated herein by reference; and plasmids and kits available from ATCC and Cell Biolabs, Inc.). In some embodiments, a nucleic acid (e.g., a vector, such as a plasmid) comprising a transgene of interest may be combined with one or more helper nucleic acids (e.g., a vector, such as a plasmid), e.g., that contain a rep gene (e.g., encoding Rep78, Rep68, Rep52 and Rep40) and a cap gene (encoding VP1, VP2, and VP3, including a modified VP region as described herein), and transfected into a recombinant cells, called helper or producer cells, such that the nucleic acid vector is packaged or encapsidated inside the capsid and subsequently purified.

Non-limiting examples of mammalian helper cells include HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). A non-limiting example of an insect helper cells is Sf9 cells (see, e.g., ATCC® CRL-1711™). In some embodiments, a helper cell may comprise rep and/or cap genes that encode the Rep protein and/or Cap proteins. In some embodiments, the packaging is performed in vitro (e.g., outside of a cell).

In some embodiments, a nucleic acid vector (e.g., a plasmid) containing the transgene of interest (e.g., SERPINA1, TTR, ATP7B or OTC) is combined with one or more helper plasmids, e.g., that contain a rep gene of a first serotype and a cap gene of the same serotype or a different serotype, and transfected into helper cells such that the rAAV particle is packaged. In some embodiments, the one or more helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins, such as E1a, E1b, E4, E2a, and VA. Helper plasmids, and methods of making such plasmids, are known in the art and commercially available (see, e.g., pDF6, pRep, pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, J. Virol., Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also know and available. For example, pSub201 is a plasmid that comprises the coding regions of the wild-type AAV2 genome (Samulski et al. (1987), J Virology, 6:3096-3101).

Inverted terminal repeat (ITR) sequences and plasmids containing ITR sequences are known in the art and are commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996 November; 93(24):14082-7; and Curtis A. Machida. Methods in Molecular Medicine™. Viral Vectors for Gene Therapy Methods and Protocols. 10.1385/1-59259-304-6:201 ® Humana Press Inc. 2003. Chapter 10. Targeted Integration by Adeno-Associated Virus. Matthew D. Weitzman, et al.; U.S. Pat. Nos. 5,139,941 and 5,962,313, all of which are incorporated herein by reference).

Genbank reference numbers for sequences of AAV serotype 3B are listed in patent publication WO 2012/064960, which is incorporated herein by reference in its entirety.

A non-limiting method of rAAV particle production method is described next. In some embodiments, one or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. In some embodiments, the cap ORF may also comprise one or more modifications to produce a modified capsid protein as described herein. As an example, in some embodiments, HEK293 cells (available from ATCC®) are transfected via CaPO₄-mediated transfection, lipids or polymeric molecules such as Polyethylenimine (PEI) with the helper plasmid(s) and a plasmid containing a nucleic acid vector. In some embodiments, the HEK293 cells are then incubated for at least 60 hours to allow for rAAV particle production. Alternatively, in some embodiments, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus (e.g., Ad5) and herpesvirus.

Alternatively, in some embodiments, Sf9-based producer stable cell lines are infected with a single recombinant baculovirus containing the nucleic acid vector. In some embodiments, HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. In some embodiments, he HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation. See US Patent Publication No. 2017/0130208, incorporated herein by reference.

Methods for large-scale production of AAV using a herpesvirus-based system are also known. See for example, Clement et al., Hum Gene Ther. 2009, 20(8):796-806. Methods of producing exosome-associated AAV, which can be more resistant to neutralizing anti-AAV antibodies, are also known (Hudry et al., Gene Ther. 2016, 23(4):380-92; Macguire et al., Mol Ther. 2012, 20(5):960-71).

Methods for producing and using pseudotyped rAAV vectors are also known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Illustrative embodiments of the present disclosure are described below. In the interest of clarity, not all features of an actual implementation are described in this specification. It will be appreciated by one of skill in the art that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would be a routine undertaking for those of ordinary skill in the art having the benefit of this disclosure.

### Exemplary Definitions

Unless otherwise defined, all technical terms used herein have the same meaning as commonly understood by one of ordinary skill in the art to which this present disclosure belongs. Commonly understood definitions of molecular biology terms can be found in Rieger et al., (1991) and Lewin (1994). Commonly understood definitions of virology terms can be found in Granoff and Webster (1999) and Tidona and Darai (2002).

In accordance with convention, the words “a” and “an” when used in this application, including the claims, denotes “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

The term “e.g.,” as used herein, is used merely by way of example, without limitation intended, and should not be construed as referring only those items explicitly enumerated in the specification.

As used herein, “an effective amount” would be understood by those of ordinary skill in the art to provide a therapeutic, prophylactic, or otherwise beneficial effect against the organism, its infection, or the symptoms of the organism or its infection, or any combination thereof.

The phrase “expression control sequence” refers to any genetic element (e.g., polynucleotide sequence) that can exert a regulatory effect on the replication or expression (transcription or translation) of another genetic element. Common expression control sequences include promoters, polyadenylation (polyA) signals, transcription termination sequences, upstream regulatory domains, origins of replication, internal ribosome entry sites (IRES), enhancers, and the like. A “tissue specific expression control sequence” is one that exerts a regulatory effect on the replication or expression (transcription or translation) of another genetic element in only one type of tissue or a small subset of tissues.

The phrase “helper function” is meant as a functional activity performed by a helper nucleic acid or polypeptide encoded by a helper nucleic acid that is derived from a virus, such as Adenovirus (Ad) or herpesvirus (e.g., a recombinant HSV) and that facilitates AAV replication in a host cell. In some embodiments, a helper nucleic acid provides helper function by encoding one or more of the E1, E2A, E4, and/or VA genes. In some embodiments, a helper nucleic acid is provided by a helper virus (e.g., Ad, such as Ad5, or a recombinant herpesvirus). Accordingly, helper functions provided by a helper nucleic acid increase the efficiency of capsid production.

The term “packaging nucleic acid” refers to a nucleic acid that encodes AAV rep and AAV cap genes (e.g., a variant AAV cap gene, such as V04 or V05).

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements. As a further example, a recombinant AAV genome may comprise a heterologous nucleic acid (e.g., a transgene or expression construct) which is heterologous to the ITRs which flank said sequence.

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity may be determined by analyzing two or more sequences using algorithms and computer programs known in the art. Such methods may be used to assess whether a given sequence is identical or homologous to another selected sequence.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the terms “humanize” and “humanized” refers to the action of engrafting human cells or tissues into a non-human animal, such as a mouse. The present disclosure may refer to humanized murine models and/or subjects, such as mouse models humanized with primary human hepatic cells.

The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, the term “in need of treatment” refers to a judgment made by a caregiver such as a physician or veterinarian that a patient requires (or will benefit in one or more ways) from treatment. Such judgment may made based on a variety of factors that are in the realm of a caregiver's expertise and may include the knowledge that the patient is ill as the result of a disease state that is treatable by one or more compound or pharmaceutical compositions such as those set forth herein.

The terms “isolated” or “biologically pure” refer to material that is substantially, or essentially, free from components that normally accompany the material as it is found in its native state. Thus, isolated polynucleotides in accordance with the present disclosure preferably do not contain materials normally associated with those polynucleotides in their natural, or in situ, environment.

As used herein, the term “kit” may be used to describe variations of the portable, self-contained enclosure that includes at least one set of components to conduct one or more of the diagnostic or therapeutic methods of the present disclosure.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “library” refers to a collection of elements that differ from one another in at least one aspect. For example, a vector library is a collection of at least two vectors that differ from one another by at least one nucleotide. As another example, a “virion library” is a collection of at least two virions that differ from one another by at least one nucleotide or at least one capsid protein.

As used herein, the term “master library” or “combined library” refers to a pool of rAAV virions composed of chimeric rcAAV nucleic acid vectors encapsidated in cognate chimeric capsids (e.g., capsids containing a degenerate or otherwise modified Cap protein). As used herein, the term “rcAAV nucleic acid vector” refers to a replication-competent AAV-derived nucleic acid capable of DNA replication in a cell without any additional AAV genes or gene products.

As used herein, the term “parent sub-library” refers to a pool of rAAV virions composed of chimeric rcAAV nucleic acid vectors encapsidated in cognate chimeric capsids (e.g., capsids containing degenerate or otherwise modified Cap protein). More than one parent sub-library may be combined to create a master library or combined library.

When referring to a nucleic acid molecule or polypeptide, the term “native” refers to a naturally-occurring (e.g., a WT) nucleic acid or polypeptide.

The terms “naturally-occurring” or “native,” as used herein refers to the fact that the described molecule may be found in nature. For example, a polypeptide or polynucleotide sequence that is present in an organism (including viruses) that may be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, the phrase “nucleic acid” means a chain of two or more nucleotides such as RNA (ribonucleic acid) and DNA (deoxyribonucleic acid). Conventional nomenclature exists in the art for polynucleotide and polypeptide structures. For example, one-letter abbreviations are widely employed to describe nucleotides: Adenine (A), Guanine (G), Cytosine (C), Thymine (T), Uracil (U), Purine, i.e. A or G (R), Pyrimidine, i.e. C or T (Y), any nucleotide (N), Weak, i.e. A or T (W), Strong, i.e. G or C (S), Amino, i.e. A or C (M), Keto, i.e. G or T (K), not A, i.e. G or C or T (B), not G, i.e. A or C or T (H), not C, i.e. A or G or T (D) and not T, i.e. A or G or C (V).

In accordance with the present disclosure, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

The phrases “cap nucleic acid,” “cap gene,” and “capsid gene” as used herein mean a nucleic acid that encodes a Cap protein. Examples of cap nucleic acids include “wild-type” (WT) Cap-encoding nucleic acid sequences from AAV serotypes 1, 2, 3, 4, 5, 6, 7, 8, 9, 10, 11, 12, 13; a native form cap cDNA; a nucleic acid having sequences from which a cap cDNA can be transcribed; and/or allelic variants and homologs of the foregoing.

“VR”, “VRs”, “variable region” or “variable regions” refer to amino acid stretches of capsid protein that do not have a high degree of homology between AAV variants. These amino acid stretches are commonly designated as VRs I through IX (also known as “loops”).

VRs are localized at the surface of the assembled capsid and interact with host cell surface receptors and other host factors.

The phrase “pharmaceutically acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (e.g., nucleic acids) and capable of transporting another nucleic acid to which it has been linked. Typically, a plasmid or a vector contains an origin of replication that is functional in bacterial host cells, e.g., Escherichia coli, and selectable markers for detecting bacterial host cells including the plasmid. Plasmids and vectors of the present disclosure may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources. One type of preferred vector is an episome, e.g., a nucleic acid capable of extra-chromosomal replication. Preferred vectors are those capable of autonomous replication and/or expression of nucleic acids to which they are linked.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Additional conventions include: Asn or Asp (B; Asx), Gln or Glu (Z; Glx), Leu or Ile (J; Xle), Selenocysteine (U; Sec), Pyrrolysine (O; Pyl) and Unknown (X; Unk). Amino acid residues described herein are preferred to be in the “L” isomeric form. However, residues in the “D” isomeric form may be substituted for any L-amino acid residue provided the desired properties of the polypeptide are retained.

The term “promoter,” as used herein refers to a region or regions of a nucleic acid sequence that regulates transcription of an operably linked gene.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from two to about 20 amino acid residues in length, oligopeptides from about 10 to about 100 amino acid residues in length, and longer polypeptides including those of about 100 or more amino acid residues in length. Furthermore, the term is also intended to include enzymes, i.e., functional biomolecules including at least one amino acid polymer. Polypeptides and proteins of the present disclosure also include polypeptides and proteins that are or have been post-translationally modified, and include any sugar or other derivative(s) or conjugate(s) added to the backbone amino acid chain.

The term “pseudotyped” is meant a nucleic acid or genome derived from a first AAV serotype that is encapsidated (packaged) into an AAV capsid containing at least one AAV Cap protein of a second serotype differing from the first serotype.

The term “recombinant” indicates that the material (e.g., a polynucleotide or a polypeptide) has been artificially or synthetically (non-naturally) altered by human intervention. The alteration may be performed on the material within or removed from, its natural environment or state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced to comprise a recombinant genome comprising a heterologous nucleic acid flanked by ITRs.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The terms “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75% sequence identity as compared to a selected reference nucleic acid or amino acid sequence. More typically, the selected sequence and the reference sequence will have at least about 80, 81, 82, 83, 84 or even 85% sequence identity, and more preferably, at least about 86, 87, 88, 89, 90, 91, 92, 93, 94, or 95% sequence identity. More preferably still, highly homologous sequences often share greater than at least about 96, 97, 98, or 99% sequence identity between the selected sequence and the reference sequence to which it was compared.

The percentage of sequence identity may be calculated over the entire length of the sequences to be compared or may be calculated by excluding small deletions or additions which total less than about 25% or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

As used herein, the term “structural gene” is intended to generally describe a polynucleotide, such as a gene, that is expressed to produce an encoded peptide, polypeptide, protein, ribozyme, catalytic RNA molecule, or antisense molecule.

As used herein, the term “spheroid” refers to a three-dimensional spherical cellular aggregate culture model. Spheroids (e.g. hepatospheres) may better simulate a live cell's environmental conditions compared to a two-dimensional culture model, specifically with respect to reactions between cells.

The term “subject,” as used herein, describes an organism, including a mammal such as a human primate, to which treatment with one or more of the disclosed compositions may be provided. Mammalian species that may benefit from the disclosed treatment methods include, without limitation, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. The term “host” refers to any host organism that may receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner.

As used herein, the terms “terminal repeat”, “inverted terminal repeat” or “ITR” refer to a nucleic acid sequence derived from an AAV that is required in cis for replication and packaging of AAV.

“Transcriptional regulatory element” refers to a polynucleotide sequence that activates transcription alone or in combination with one or more other nucleic acid sequences. A transcriptional regulatory element may include, for example, one or more promoters, one or more response elements, one or more negative regulatory elements, one or more enhancers, or any combination thereof.

As used herein, a “transcription factor recognition site” and a “transcription factor binding site” refer to a polynucleotide sequence(s) or sequence motif(s) that are identified as being sites for the sequence-specific interaction of one or more transcription factors, frequently taking the form of direct protein-DNA binding. Typically, transcription factor binding sites may be identified by DNA footprinting, gel mobility shift assays, and the like, and/or may be predicted based on known consensus sequence motifs, or by other methods known to one of ordinary skill in the relevant molecular biological and virology arts.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

As used herein, the term “transformed cell” is intended to mean a host cell whose nucleic acid complement has been altered by the introduction of one or more exogenous polynucleotides into that cell.

As used herein, the term “transformation” is intended to generally describe a process of introducing an exogenous polynucleotide sequence (e.g., a viral particle, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the terms “treat,” “treating,” and “treatment” refer to the administration of a composition to reduce the frequency or severity of at least one sign or symptom of a disease, disorder or condition experienced by a subject. These terms embrace prophylactic administration, i.e., prior to the onset of clinical symptoms of a disease state so as to prevent any symptom or characteristic of the disease state. The disclosed compositions may be administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell. In some embodiments, the disease, disorder or condition is AAT, FAP, OTC Deficiency, or Wilson's Disease. Such treating need not be absolute to be deemed medically useful. As such, the terms “treatment,” “treat,” “treated,” or “treating” may refer to therapy, or the amelioration or reduction in the extent or severity of disease, disorder or condition, of one or more symptom thereof, whether before or after onset of the disease, disorder or condition.

As used herein, the term “rAAV nucleic acid vector” is a recombinant AAV-derived nucleic acid containing a heterologous nucleic acid (e.g., a transgene or expression construct) flanked by ITRs.

The terms “virion” and “particle”, such as rAAV virions and rAAV particles, are used interchangeably and refer to particle formed by AAV capsid proteins. In some embodiments, a virion or a particle comprises a nucleic acid encapsidated by a capsid comprising at least one AAV capsid protein. In some embodiments, the capsid protein is a variant AAV capsid protein described herein. Accordingly, an “rAAV virion” can be a virion that includes nucleic acid sequences (e.g., one comprising a heterologous nucleic acid, such as a transgene or an expression construct, flanked by AAV ITRs) and/or proteins derived from a rAAV expression construct.

As used herein, the term “tropism” refers to the cells and/or tissues of a host which support growth of a particular serotype of AAV. Some serotypes may have a broad tissue tropism and can infect many types of cells and tissues. Other serotypes may infect primarily a single tissue or cell type.

As used herein, the term “variant” refers to a molecule (e.g., a capsid) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wildtype capsid. Variants of a protein molecule, e.g. a capsid, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the capsid protein. These modifications include chemical modifications as well as truncations.

By a protein (e.g., a capsid protein) comprising an amino acid sequence having at least, for example, 95% “identity” to a query amino acid sequence, it is intended that the amino sequence of the subject amino acid molecule is identical to the query sequence except that the subject amino acid molecule sequence may include up to five amino acid alterations per each 100 amino acids of the query sequence. In other words, to obtain a capsid having an amino sequence at least 95% identical to a reference (query) sequence, up to 5% of the amino acids in the subject sequence may be inserted, deleted, or substituted with another nucleotide. These alterations of the reference sequence may occur at the N- or C- terminus of the reference sequence or anywhere between those positions, interspersed either individually among amino acids in the reference sequence or in one or more contiguous groups within the reference sequence.

As a practical matter, whether any particular amino acid molecule is at least 80%, 85%, 90%, 95%, 96%, 97%, 98%, or 99% identical to, for instance, the amino acid sequence of a capsid protein, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either amino acid sequence or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the N- or C- terminus, relative to the query sequence, the percent identity is corrected by calculating the number of nucleotides of the query sequence that are positioned N- or C- terminus to the query sequence, which are not matched/aligned with a corresponding subject nucleotide, as a percent of the total bases of the query sequence.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

### Step 1: Sub-Libraries Assembly.

Using pITR3-R3C3-AatII as a template, the following ten PCR reactions were conducted:

A3CL-A (VRs-I, V, VI):

| | | PCR fragment |
|---|---|---|
| | Primers | size |
| 1. | A3CL-F + A3CL-A1R (before VR-I) | 86 bp |
| 2. | A3CL-A1F + A3CL-A2R (VR-I to most of VR-V) | 747 bp |
| 3. | A3CL-A2F + A3CL-A3R (part of VR-V to VR-VI) | 136 bp |
| 4. | A3CL-A3F + A3CL-R (after VR-VI) | 281 bp |

A3CL-B (VR-IV):

| | Primers | PCR fragment size |
|---|---|---|
| 5. | A3CL-F + A3CL-B1R (before VR-IV) | 647 bp |
| 6. | A3CL-B1F + A3CL-R (VR-IV to end) | 556 bp |

A3CL-C (VR-VII):

| | Primers | PCR fragment size |
|---|---|---|
| 7. | A3CL-F + A3CL-C1R (before VR-VII) | 935 bp |
| 8. | A3CL-C1F + A3CL-R (VR-VII to end) | 266 bp |

A3CL-D (VR-VIII):

| | Primers | PCR fragment size |
|---|---|---|
| 9. | A3CL-F + A3CL-D1R (before VR-VIII) | 1055 bp |
| 10. | A3CL-D1F + A3CL-R (VR-VIII to end) | 147 bp |

The respective PCR fragments were eluted from the agarose gel, mixed at equimolar ratios as indicated above for sub-libraries A, B, C, and D, and subjected to 15 cycles of overlap extension (OE) without primers, followed by 20 cycles of PCR using A3CL-F forward and A3CL-R reverse primers. The resulting fragments of 1140 bp for each of the A (I+V+VI), B (IV), C (VII), or D (VIII) sub-libraries were purified on agarose gel and eluted in small volume H2O. Using isothermal DNA assembly protocol, the respective fragments were individually sub-cloned into gel-purified pTR3-R3C3-AatII digested with AatII+ApaI. Four plasmid libraries A, B, C, and D, incorporating the respective VRs were derived. The estimated plasmid libraries' complexities were the following: A—4.4×10⁷; B—1.7×10⁷; C —1×10⁸; D—1×10⁸.

### Step 2: Pre-Selecting Structurally Compatible Parent Viral Libraries.

Using plasmid libraries from Step 1, viral sub-libraries A, B, C, and D were packaged, AAV virus from each preparation was purified using iodixanol density gradients, and the viral DNAs were isolated. Next, using viral DNAs as templates, the following PCR reactions were conducted:
1. VR-I, primers A3CL-F+VR-I_IV-R, template A, size 644 bp.
2. VR-IV, primers VR-I_IV-F+VR-IV_V-R, template B, size 145 bp.
3. VR-V+VI, primers VR-IV_V-F+VR-VI_VII-R, template A, size 194 bp.
4. VR-VII, primers VR-VI_VR-VII-F+A3CL-R, template C, size 274 bp.
5. VR-VIII, primers A3CL-F and A3CL-R, template D, size 1140 bp.

The respective PCR fragments were gel-purified and used as the templates in the OE/PCR to derive two PCR fragments, each of 1140 bp: A+B+C (VR-I, IV, V, VI, VII) and D (VR-VIII).

### Step 3: Packaging Master Libraries.

Using isothermal DNA assembly protocol, the respective fragments were individually sub-cloned into gel-purified pTR3-R3C3-AatII digested with AatII+ApaI. The estimated plasmid library A+B+C complexity was 2.5×10⁷, plasmid library D complexity was 4×10⁷. Using these plasmid libraries, two final master viral libraries were packaged: ABC, with the titer of 5.7×10¹² DNase resistant particles per milliliter (DRP/ml), and D, with the titer of 8.7×10¹² DRP/ml. The assembly flowchart is shown in FIG. 5.

TABLE 1
Theoretical (calculated) complexities of A3CL for individual
VRs and combinations of VRs. The VRs and VRs combinations
constructed as sub-libraries are shown in bold font.

| VR | Complexity |
|---|---|
| I | 72 |
| IV | 2.1 × 10⁶ |
| V | 27,648 |
| VI | 144 |
| VII | 4 × 10⁷ |
| VIII | 5.44 × 10⁸ |
| I + V | 1.99 × 10⁶ |
| I + VI | 1.04 × 10⁴ |
| I + V + VI | 2.87 × 10⁸ |
| V + VI | 3.98 × 10⁶ |
| I + IV + V + VI + VII | 2.37 × 10²² |
| I + IV + V + VI + VII + VIII | 1.29 × 10³¹ |

TABLE 2
Theoretical (calculated) complexities of
constructed sub-libraries A, B, C, and D.

| Sub-library | VRs | Complexity |
|---|---|---|
| A3CL-A | I − V − VI | 2.9 × 10⁸ |
| A3CL-B | IV | 2 × 10⁶ |
| A3CL-C | VII | 4 × 10⁷ |
| A3CL-D | VIII | 5.4 × 10⁸ |

Table 3. Synthetic oligonucleotides used to assemble the AAV3B capsid library (bolded positions indicate nucleotides which may differ relative to corresponding nucleotide positions in a nucleic acid encoding the wildtype VP1 protein of serotype AAV3B and are annotated according to the IUPAC nucleotide code).

| Name | Sequence |
|---|---|
| A3CL-F | GGCTGGGCGACAGAGTCATC (SEQ ID NO: 20 |
| A3CL-A1R | GCTGGAGATTTGCTTGTAGAGATG (SEQ ID NO: 21) |
| A3CL-A1F | CATCTCTACAAGCAAATCTCCAGCVVMDCAGGAGCTASCAACGACAACCACTACTTTGGC |
| | (SEQ ID NO: 22) |
| A3CL-A2R | CCAAGGAAASTYACTGTTGTTGTTSYSGBYGKVGRYTKTIGAAAGTCTCTGTTGCC |
| | (SEQ ID NO: 23) |
| A3CL-A2F | AACAACAACAGTRASTTTCCTTGGMCAGCGGCCAGCAMATATCATCTCAATG |
| | (SEQ ID NO: 24) |
| A3CL-A3R | GATTGCCGTGCATAGGGAAAAATYTSYCSKYATCGTCCYYGTGACTGGCCATAGCTGG |
| | (SEQ ID NO: 25) |
| A3CL-A3F | ATTTTTCCCTATGCACGGCAATC (SEQ ID NO: 26) |
| A3CL-R | CATCCGTGTGAGGAATCTTTGC (SEQ ID NO: 27) |
| A3CL-B1R | TTGCGTTCTGTTCAGGTAGTACAGA (SEQ ID NO: 28) |
| A3CL-B1F | CTGTACTACCTGAACAGAACGCAARGCAMCVCNRGCGGAACARCCRVCMHSMRSVVSCTG |
| | VNGTTTAGCCAGGCTGGGCC (SEQ ID NO: 29) |
| A3CL-C1R | TTTGCCAAATATTAGATTGCC (SEQ ID NO: 30) |
| A3CL-C1F | CGGCAATCTAATATTTGGCAAASAARRCRSCRVSRVARVCRATRYCGMSDWCGRSVRS |
| | GTAATGATTACGGATGAAGAAG (SEQ ID NO: 31) |
| A3CL-D1R | CTGCAAGTTATTTGCCACAGTTC (SEQ ID NO: 32) |
| A3CL-D1F | GAACTGTGGCAAATAACTTGCAGRVSVVSMRSRVCVVSCCCACGDHTVVSRNSGTC |
| | VMSCATCAGGGGGCCTTACCTG (SEQ ID NO: 33) |
| VR-I_IV-F | CAGTATCTGTACTACCTGAACAGAACGC (SEQ ID NO: 34) |
| VR-I_IV-R | GCGTTCTGTTCAGGTAGTACAGATACTG (SEQ ID NO: 35) |
| VR-IV_V-F | CCTGGGCCCTGCTACCGGCAACAGAG (SEQ ID NO: 36) |
| VR-IV_V-R | CTCTGTTGCCGGTAGCAGGGCCCAGG (SEQ ID NO: 37) |
| VR-VI_VII-F | CCCTATGCACGGCAATCTAATATTTGGC (SEQ ID NO: 38) |
| VR-VI_VII-R | GCCAAATATTAGATTGCCGTGCATAGGG (SEQ ID NO: 39) |

Next generation (NGS) sequencing.
Number of sequences processed: 1817050
Number of distinct sequences (complexity): 1708473 (0.94)

Copy number distribution:

| Copy number | Number of sequences |
|---|---|
| 1 | 1603700 |
| 2 | 101430 |
| 3 | 3257 |
| 4 | 83 |
| 5 | 2 |
| 377 | 1 |

Table 4 shows examples of representative variants within VRs IV, V, VI, and VII from the master viral library ABC as deduced from the NGS sequencing. The left most column recites SEQ ID NOs for each VR sequences to the right of said left column. Under “450”, this column shows amino acid residue positions 450-462 of the VP1 protein of serotype AAV3B which correspond to the VR IV thereof. Under “491”, this column shows amino acid residue positions 491-508 of the VP1 protein of serotype AAV3B which correspond to the VR V thereof. Under “528”, this column shows amino acid residue positions 528-533 of the VP1 protein of serotype AAV3B which correspond to the VR VI thereof. Under “546”, this column shows amino acid residue positions 546-557 of the VP1 protein of serotype AAV3B which correspond to the VR VII thereof. Each column is separated by a space to indicate that the sequences of each respective column are not contiguous and instead represent discreet VRs. Sequences on lines 1-86 (listed top to bottom) correspond to the SEQ ID NOs shown in the left most column of the table wherein the SEQ ID NOs read left to right correspond to a respective VR Dots shown horizontally indicate wildtype amino acid residue positions wherein each dot is a respective amino acid residue position. n line 1, “wildtype” refers to the natural amino acid sequence of each indicated VR of AAV3B VP1 protein which is fully disclosed above. Thus, line 1 consists of horizontal dots to show all positions therein consist of wildtype amino acid residues. These sequences in lines 2-86 show the amino acid sequence variants of each VR relative to wildtype in line 1. For example, SEQ ID NOs 45-48 correspond to a VR IV of SEQ ID NO: 45, a VR V of SEQ ID NO: 46, a VR VI of SEQ ID NO: 47, and a VR VII of SEQ ID NO: 48. For capsid variants (lines numbered 2-86) amino acid residue positions that differ relative to wildtype VR (line 1) are shown.

TABLE 4
Representative Variants of AAV capsid VP1 VRs IV, V, VI From Master Viral Library ABC

| | Wild | | | | | | |
|---|---|---|---|---|---|---|---|
| SEQ ID NOs. | type | 450 | 491 | 528 | 546 | cn | % |
| 41-44 | | GTTSGTTNQSRLL | KTANDNNNSNFPWTAASK | KDDEEK | EGTTASNAELDN | | |
| 41-44 | 1 | ............. | .................. | ...... | ............ | 377 | 0.0 |
| 45-48 | 2 | .N.G...SP...R | .IYDR............T | G..TGR | Q..GEG.V.VGK | 5 | 0.0 |
| 49-52 | 3 | S......G.RK.A | .AYGH....D...P...T | G...DR | QDSGENDVAIGR | 5 | 0.0 |
| 53-56 | 4 | ..A....AN.N.K | ..YS.............. | G..DDR | ...DGA.V.I.R | 4 | 0.0 |
| 57-60 | 5 | ..P...AAHKT.E | ..SAE............. | G..AGR | .DAEGGD.AIGG | 4 | 0.0 |
| 61-64 | 6 | S.AG..AT.KA.T | .VHAH............. | E..TGR | QDA.R..VAFEE | 4 | 0.0 |
| 65-68 | 7 | .NP....GLRG.T | T.D.E........P.... | ...AG. | Q..DGN.IAFGE | 4 | 0.0 |
| 69-72 | 8 | .N.....SKRP.M | T...E............T | E..N.R | .DAKGTDT.F.R | 4 | 0.0 |
| 73-76 | 9 | SNA....GIHQ.K | TAPDR....E........ | E..NGR | QNGATADT.VER | 4 | 0.0 |
| 77-80 | 10 | SN.G...AMRE.E | .AP......K.......T | E..TG. | .S.AETDV.DGR | 4 | 0.0 |
| 81-84 | 11 | SNA...AGLQ..K | .IPDQ............. | ...NG. | QSGG.ADIDNG. | 4 | 0.0 |
| 85-88 | 12 | .NP....APH... | TIH.G........P...T | ...DGR | QDGGT..IDI.G | 4 | 0.0 |
| 89-91, 44 | 13 | ..P....DLRE.A | .IP..............T | E...DR | ............ | 4 | 0.0 |
| 92-95 | 14 | SNP...A.PRT.M | .IDAH....E...P...T | E..NG. | QSS.TGDV.D.D | 4 | 0.0 |
| 96-99 | 15 | .NA....DTK..T | .ASGG............T | ...DD. | ..SNRDD..V.R | 4 | 0.0 |
| 100-103 | 16 | .N.G...DIR..R | ..HSE....E.......T | ...N.R | QD.RETDVAI.R | 4 | 0.0 |
| 104-107 | 17 | .NA...AGMRE.M | .A..H............. | E...D. | .SGS.DDVAIGR | 4 | 0.0 |
| 108, 109, | 18 | SN.G..ATPKQ.Q | .ASAH....E........ | .....R | ..S.RNDIANEH | 4 | 0.0 |
| 9, 110 | | | | | | | |
| 111-114 | 19 | SN.G..A.IKE.T | ..S..............T | R..ND. | QSASKNDI.YEQ | 4 | 0.0 |
| 115-118 | 20 | SNAG...SNRE.R | T.SSQ............T | R..DDR | QDAGGNDV.VGD | 4 | 0.0 |
| 119-122 | 21 | SN....ATT.A.K | ..YGH............. | G..T.. | Q.GS.N.V.VES | 4 | 0.0 |
| 123, 46, | 22 | SNAG..AATN... | .IYDR............T | R...D. | ..GEKG.VDI.R | 4 | 0.0 |
| 124, 125 | | | | | | | |
| 126-129 | 23 | S.P....ATKG.T | TAHTG............. | G..DG. | ..S..TDVAIGS | 4 | 0.0 |
| 130-133 | 24 | .N.G...DLR..M | T.D.H....E...P...T | G..KGR | .NGAKNDIAFEG | 4 | 0.0 |
| 134, 135, | 25 | S......TLKA.Q | .IP.R............T | G...DR | .NSKGA.T.I.E | 4 | 0.0 |
| 51, 136 | | | | | | | |
| 137, 138, | 26 | .......DPKD.V | T.HG.....D.......T | ...DD. | .D.A.D.V.FGR | 4 | 0.0 |
| 98, 139 | | | | | | | |
| 140-143 | 27 | S.AG...TIKD.V | .VPD.....K........ | E..D.R | QDSG.T.V.FGR | 4 | 0.0 |
| 144-147 | 28 | ...G...TMRK.G | .VYGG............T | E..A.R | QSSGRNDV.YGD | 4 | 0.0 |
| 148-151 | 29 | .N.G..ASTR..T | .IPDQ........P.... | E...GR | QSAEKGDI.YGR | 4 | 0.0 |
| 152-155 | 30 | .N.....ATHT.A | .IHSR....D...P.... | E..AG. | Q.A..G.IDVEQ | 4 | 0.0 |
| 156-159 | 31 | SNPG...SIRG.Q | TIP.R........P...T | R..TD. | Q.GG.G.TDF.H | 4 | 0.0 |
| 160-163 | 32 | S.....AAPRG.V | TVYGH....E........ | ....GR | ..AG...VAIEE | 4 | 0.0 |
| 164-167 | 33 | .NA...ATKQG.M | .VP.Q....D........ | ...DDR | QSSDKN...D.S | 4 | 0.0 |
| 168-171 | 34 | SNAG..ATT.Q.R | TAPAE........P...T | R..ADR | .SGRGD.VDFEK | 4 | 0.0 |
| 172-175 | 35 | SN.G..AGIRA.Q | .VDTG....D.......T | E..T.. | .NSARND.DIGR | 4 | 0.0 |
| 176-179 | 36 | .NA...AA.NG.R | .IP.E....K.......T | ....G. | .SSSGDD..FGG | 4 | 0.0 |
| 180, 181, | 37 | SN....AGPQQ.R | ..HAQ............. | E..TG. | ..AR.NDIAF.Q | 4 | 0.0 |
| 79, 182 | | | | | | | |
| 183-186 | 38 | S.P....SMRT.E | .APAR....E.......T | R..AG. | Q.SRENDT.F.G | 4 | 0.0 |
| 187-190 | 39 | S.AG...ALKG.K | TI.DH....E...P...T | R..K.. | .DS.GA.IAD.R | 4 | 0.0 |
| 191-194 | 40 | S.P...ASTRT.M | ..H.H....E.......T | ....D. | ...E.T.VAIGG | 4 | 0.0 |
| 195, 196, | 41 | .NPG....NQA.R | .IHGQ....D.......T | R..ND. | ..SARGDVAYEK | 4 | 0.0 |
| 113, 197 | | | | | | | |
| 198, 199, | 42 | SNA....DTRE.V | TI.D.....E.......T | R..TD. | Q.SAGADV.VEK | 4 | 0.0 |
| 158, 200 | | | | | | | |
| 201-204 | 43 | SNPG....LRE.R | TIHTE....E.......T | R..KDR | Q.GGGT.V.IGS | 4 | 0.0 |
| 205-208 | 44 | ...G..A.NNT.. | .I.SG............T | ...KGR | ..AEKNDTAVG. | 4 | 0.0 |
| 209, 210, | 45 | .......DKQQ.M | ..H.G....D........ | E..TG. | QSAEGN.VAY.G | 4 | 0.0 |
| 79, 211 | | | | | | | |
| 212-215 | 46 | ..AG..ATL.T.V | .ISAG....D........ | G..NG. | QNS...DVAI.G | 4 | 0.0 |
| 216, 217, | 47 | SN....AGLRT.T | .ADA.....D...P...T | G..NG. | .DASGN.V.DGR | 4 | 0.0 |
| 214, 218 | | | | | | | |
| 219-222 | 48 | SNA...ATP.T.R | ..DTH....E...P.... | R..NDR | ..ARG..IDVGD | 4 | 0.0 |
| 223-226 | 49 | S.A...ASLRA.M | .VP.R............T | G..ND. | .NAR..D..V.R | 4 | 0.0 |
| 227-230 | 50 | ..A...ATTKG.. | .ISTQ............. | E..AD. | Q.GETD.VDVGD | 4 | 0.0 |
| 231-234 | 51 | ..A...AALKQ.A | .ADS.............T | E..ADR | Q.GETG.I.Y.G | 4 | 0.0 |
| 235-238 | 52 | .NA...ATT.N.M | .ADDR............T | ....DR | Q.AKR.DTAVEE | 4 | 0.0 |
| 239-242 | 53 | ..AG..A.MKD.R | T..SE....D.......T | E..KD. | ..ANGGDVAIGQ | 4 | 0.0 |
| 243, 244, | 54 | S.PG...TIRD.K | TVST.....D...P...T | ...DDR | ..SGRN.VAVEE | 4 | 0.0 |
| 166, 245 | | | | | | | |
| 246, 247, | 55 | S.P...A.INT.R | ..P.R............T | E...GR | QSA.KDDVDIGG | 4 | 0.0 |
| 150, 248 | | | | | | | |
| 249-252 | 56 | .N.G..AGLQK.M | ..HGG........P...T | ...DG. | QSSRGNDVAV.D | 4 | 0.0 |
| 253, 254, | 57 | .N.....TPRT.A | .IPSH....E........ | ...... | Q.SNG..I.FGS | 4 | 0.0 |
| 43, 255 | | | | | | | |
| 256, 257, | 58 | SNAG..AGLRQ.T | .APAE....D.......T | E..AG. | ..GGGA.IAVEE | 4 | 0.0 |
| 154, 258 | | | | | | | |
| 259-262 | 59 | ..A...AAK.T.V | .ISTR............T | E..... | ...SKNDV.VE. | 4 | 0.0 |
| 263-266 | 60 | .......TTR..M | .IYGG............T | E..AGR | Q..ATA.V.VES | 4 | 0.0 |
| 267, 268, | 61 | ..AG..AGMRE.A | TIYTG............. | ....GR | ..SSTGD.DVGR | 4 | 0.0 |
| 162, 269 | | | | | | | |
| 270, 271, | 62 | S..G..A.PKE.R | TA..H....E.......T | E...D. | Q.AGE..VAI.G | 4 | 0.0 |
| 106, 272 | | | | | | | |
| 273-276 | 63 | .......GT.T.R | ..DTG............. | G....R | ..AGTAD.AV.G | 4 | 0.0 |
| 277-280 | 64 | .NAG....KRD.. | TAYTR....D.......T | ...D.. | Q..GKTD.DNGG | 4 | 0.0 |
| 281-284 | 65 | .NA....DMKH.T | .ISDR........P...T | R..N.R | QS.RGG...I.G | 4 | 0.0 |
| 285-288 | 66 | SN.G..ADLRD.. | TIPTQ....E........ | ...NDR | QSAK.NDV.V.R | 4 | 0.0 |
| 289, 290, | 67 | S.A....ATQQ.V | T.DSQ....E.......T | R..NDR | .NAEGG.V.IGQ | 4 | 0.0 |
| 221, 291 | | | | | | | |
| 292-295 | 68 | .NAG...ANKT.M | .I.AH....E.......T | R..DG. | QDSS.D.I.YGK | 4 | 0.0 |
| 296, 297, | 69 | .NAG...GTKE.R | TI..E............T | ...D.. | .DAKRN.VDY.G | 4 | 0.0 |
| 279, 298 | | | | | | | |
| 299, 300, | 70 | SNP....GK.S.K | ..S.E............. | ...NG. | .DSR.GD.DFEK | 4 | 0.0 |
| 83, 301 | | | | | | | |
| 302-305 | 71 | .N....ASIRQ.Q | ..PDG....K........ | R..NGR | ..S.EG.I.IEG | 4 | 0.0 |
| 306, 307, | 72 | .N.G...TL.A.G | TAHTQ............. | R..ND. | QSS.GGDTAF.G | 4 | 0.0 |
| 113, 308 | | | | | | | |
| 309-312 | 73 | SNP....TTQ..Q | ..D......D...P...T | R..TDR | ..S.GGD..IER | 4 | 0.0 |
| 313-316 | 74 | SNA....TMRK.G | TISSG........P.... | G..N.. | QDSSENDVADER | 4 | 0.0 |
| 317, 318, | 75 | S.AG..ATMQ..M | T.DTG............T | R..N.R | Q.GEGGDI.D.R | 4 | 0.0 |
| 283, 319 | | | | | | | |
| 320-323 | 76 | ..AG..ATTRD.Q | T.DDH....D........ | ...NGR | Q.GRGA.TAYEG | 4 | 0.0 |
| 324, 325, | 77 | ...G..AAM.A.R | T.DDG....K...P.... | ...... | .DGGT...AIGD | 4 | 0.0 |
| 43, 326 | | | | | | | |
| 327, 90, | 78 | .N.....TNRE.M | .IP..............T | R...D. | .D.GRADV.VGR | 4 | 0.0 |
| 124, 328 | | | | | | | |
| 329-332 | 79 | SNAG..ADKQD.V | TAHSE............. | E..DDR | Q.AAGGDI.VGS | 4 | 0.0 |
| 333-336 | 80 | .NA...AATHE.. | T.HDH....D........ | R..A.R | ..GAK.DVDFGS | 4 | 0.0 |
| 337-340 | 81 | SNA...ADTRH.M | T.PGE....D...P...T | G..TG. | Q.SATTDI.YGE | 4 | 0.0 |
| 341, 342, | 82 | SN.....A..K.Q | .IH.R....D.......T | R..DDR | Q.AEG.DVAVGD | 4 | 0.0 |
| 117, 343 | | | | | | | |
| 344-347 | 83 | .NP...AD.RA.Q | .IPTG....D.......T | R..T.. | Q..GG.DI.IGG | 4 | 0.0 |
| 348-351 | 84 | SNA...AGLNA.K | .AYTH....D.......T | G..D.R | .NAK.G..AI.G | 4 | 0.0 |
| 352, 353, | 85 | .NP.....LQ..M | .IDDQ........P...T | ....D. | .SGGTADVAV.K | 4 | 0.0 |
| 193, 354 | | | | | | | |
| 355, 356, | 86 | ......ASIQ..Q | ..YA.....E.......T | ....D. | .SAAG.DT.V.G | 4 | 0.0 |
| 193, 357 | | | | | | | |

Calculated plasmid library complexity based on colony count (2.5×10⁷) and NGS sequencing (0.94 of unique sequences) is 2.35×10⁷. wt AAV3 contamination is 0.02%.

| Q5 PCR: | |
|---|---|
| 50 μl: | 10 μl 5xB Q5 |
| | 0.4 μl 25 mM dNTPs |
| | 2.5 μl F |
| | 2.5 μl R |
| | 1 μl (1 ng) pITR3-R3C3-AatII |
| | 0.5 μl Q5 Pol |
| | H₂O up to 50 μl |
| 98° C. 30 sec | 30 cycles |
| 98° C. 10 sec | |
| 65° C. 20 sec | |
| 72° C. 30 sec | |
| 72° C. 2 min | |

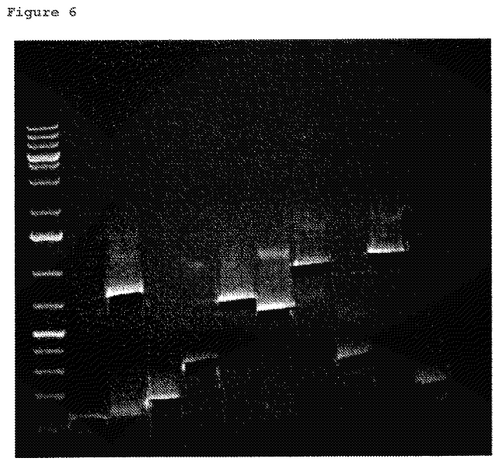

See FIG. 6.

TABLE 5

| | | Size | Conc. | | μl/5 × 10⁹ |
|---|---|---|---|---|---|
| | Fragment | (bp) | (μg/ml) | nM | copies |
| A | 1 | 86 | 10.4 | 186 | 2.2 |
| I + V + VI | 2 | 747 | 33.3 | 69 | 6 |
| | 3 | 136 | 13.2 | 149 | 2.8 |
| | 4 | 281 | 35.4 | 194 | 2.1 |
| B | 5 | 647 | 48 | 114 | 3.6 |
| IV | 6 | 556 | 29.4 | 81 | 5.1 |
| C | 7 | 935 | 22.5 | 37 | 11.2 |
| VII | 8 | 266 | 11.2 | 65 | 6.4 |
| D | 9 | 1055 | 38.8 | 57 | 7.3 |
| VIII | 10 | 147 | 35.9 | 376 | 1.1 |

TABLE 6
OE Q5 PCR

| | | | | | | | | 0 | xB | NTP | 3CL-F | 3CL-R | 5 | ₂O |
|---|---|---|---|---|---|---|---|---|---|---|---|---|---|---|
| .2 | .8 | .1 | | | | | | | 0 | .4 | .5 | .5 | .5 | 6 |
| | | | .6 | .1 | | | | | 0 | .4 | .5 | .5 | .5 | 0.4 |
| | | | | | 1.2 | .4 | | | 0 | .4 | .5 | .5 | .5 | 1.5 |
| | | | | | | | .3 | .1 | 0 | .4 | .5 | .5 | .5 | 0.7 |

1. Assays A, B, C, and D are assembled without primers, substituting H₂O for the primers' volumes (5 μl) and subjected to the following overlap extension:

| | |
|---|---|
| 98° C. 30 sec | 15 cycles |
| 98° C. 10 sec | |
| 65° C. 20 sec | |
| 72° C. 60 sec | |
| 72° C. 2 min | |

2. 40 μl each A, B, C, and D from Step 1 transferred to 10 μl containing:

| | X5 |
|---|---|
| 2.5 μl A3CL-F | 12.5 |
| 2.5 μl A3CL-R | 12.5 |
| 2 μl 5xB Q5 | 10 |
| 0.08 μl dNTPs | 0.4 |
| 0.1 μl Q5 | 0.5 |
| 2.82 μl H₂O | 14.1 |

Assays are subjected to the following PCRs:

| | |
|---|---|
| 98° C. 30 sec | 20 cycles |
| 98° C. 10 sec | |
| 59° C. 20 sec | |
| 72° C. 60 sec | |
| 72° C. 2 min | |

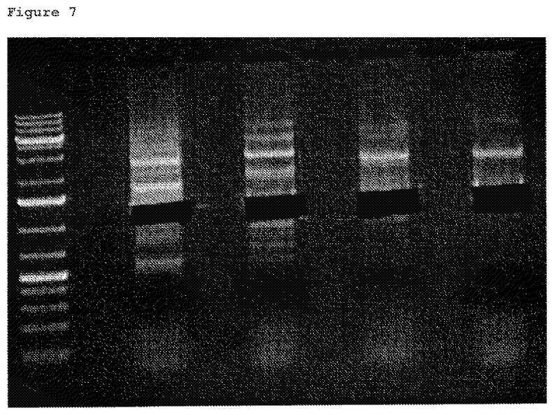

See FIG. 7: Eluted in 50 μl each A, B, C, or D; pTR3-R3C3-AatlI/AatII+ApaI eluted in 75 μl.

TABLE 7

| | | | | | | pmoles/40 μl |
|---|---|---|---|---|---|---|
| | Size | Conc. | Molarity | Conc. | μl/40 μl | assay |
| | (bp) | (ng/μl) | (nM) | (pmoles/μl) | assay | (3:1) |
| A | 1091 | 47.5 | 67 | 0.067 | 2.1 | 0.144 |
| | | | | | (100 ng) | |
| B | 1091 | 58.3 | 82 | 0.082 | 1.8 | 0.144 |
| | | | | | (100 ng) | |
| C | 1091 | 49.9 | 70 | 0.07 | 2.1 | 0.144 |
| | | | | | (100 ng) | |
| D | 1091 | 60.9 | 86 | 0.086 | 1.7 | 0.144 |
| | | | | | (100 ng) | |
| plTR3-R3C3- | 6594 | 54.3 | 13 | 0.013 | 3.7 | 0.048 |
| Aatll Aatll-Apal | | | | | (200 ng) | |
| cut | | | | | | |

| IDA | A | B | C | D | plTR3 | H₂O | |
|---|---|---|---|---|---|---|---|
| 40 μl assay:20 μl 2 × Gibson | 2.1 | | | | 3.7 | 14.2 | A |
| Master Mix (NEB)+ | | 1.8 | | | 3.7 | 14.5 | B |
| | | | 2.1 | | 3.7 | 14.2 | C |
| | | | | 1.7 | 3.7 | 14.6 | D |

Large-scale IDA for the Loop A

| | |
|---|---|
| 300 μl assay: | 150 μl 2xGibson Master Mix |
| | 27.6 μl pITR3-R3C3-AatII AatII-ApaI cut (1.5 μg) |
| | 15.8 μl A (0.75 μg) |
| | 106.6 μl H₂O |

Incubated 2 h, 50° C., Zymo-purified, eluted in 100 μl H₂O, combined with 47.5 μl of A from the pilot IDA above. Total—1.7 μg of vector plasmid DNA,
Lucigen competent cells were prepared from 4 L LB, resuspended in 8.5 ml H₂O final volume. The cell density (10 μl in 3 ml H₂O) was A₅₅₀=0.79.
Combined DNA (147.5 It) was mixed with the whole volume of competent cells and aliquoted (385 μl/aliquot, ˜10 ng plasmid DNA/50 μl competent cells) into electroporation cuvettes (total of ˜20, with outside tall electrodes) and zapped at 2.9 KV.
Cells were transferred into 1 L LB, incubated shaking at 37° C. for 1 h. Carbenicillin was added up 100 μg/ml, cell were grown at 30° C., o/n.
Total complexity from the large-scale IDA/transformation is 4.4×10⁷ clones.

Repeat IDA for the Loop C

| | |
|---|---|
| 100 μl assay: | 50 μl 2xGibson Master Mix |
| | 9.25 μl pITR3-R3C3-AatII AatII-ApaI cut (0.5 μg) |
| | 5.25 μl C (0.25 μg) |
| | 35.5 μl H₂O |

### Zymo, 50 μl H₂O.

Competent cells were prepared from 4 L LB (grown to A₅₅₀=0.6) and resuspended in a final volume 8 ml H₂O. The cell density (10 μl in 3 ml H₂O) was A₅₅₀=1.46.
180 ng vector with fragment B from the pilot IDA were electroporated with 1 ml of comp. cells, whereas 0.68 μg with fragment C—with 3 ml of cells.
After electroporation the complexity of B was ˜1.7×10⁷ (˜5 times over theoretical complexity), while C˜1×10⁸ (˜2.5 times over theoretical complexity).

TABLE 8

| | Pilot | | | | Large-scale | | | |
|---|---|---|---|---|---|---|---|---|
| | A | B | C | D | A | B | C | D |
| Complexity total | 0.9 × 10⁵ | 0.7 × 10⁵ | 1.4 × 10⁵ | 0.9 × 10⁵ | 4.4 × 10⁷ | 1.7 × 10⁷ | 1 × 10⁸ | 1 × 10⁸ |
| Volume (μl) | 400 | 400 | 400 | 400 | 1000 | 100 | 500 | 1000 |
| Complexity/μl | 225 | 175 | 525 | 225 | 4.4 × 10⁴ | 1.7 × 10⁵ | 2 × 10⁵ | 10⁵ |
| DNA concentration | 342 | 220 | 241 | 334 | 1690 | 1100 | 2100 | 2000 |
| (ng/μl) | | | | | | | | |
| Copies/μl | 4.2 × 10¹⁰ | 2.6 × 10¹⁰ | 2.9 × 10¹⁰ | 4 × 10¹⁰ | 2 × 10¹¹ | 1.3 × 10¹¹ | 2.5 × 10¹¹ | 2.4 × 10¹¹ |
| Representation | 1.9 × 10⁸ | 1.5 × 10⁸ | 0.6 × 10⁸ | 1.8 × 10⁸ | 4.5 × 10⁶ | 7.6 × 10⁵ | 1.3 × 10⁶ | 2.4 × 10⁶ |
| (copies/variant/μl) | | | | | | | | |
| Dilution factor | 42.2 | 197.4 | 46.2 | 75 | | | | |
| Final DNA | | | | | | 0.55 | 1.1 | 1 |
| concentration after | | | | | | | | |
| mixing equal | | | | | | | | |
| volumes (μg/μl) | | | | | | | | |
| Viral DNA | | | | | 27.6 | 22.8 | 23.2 | 85.5 |
| concentration, 80 | | | | | | | | |
| μl (μg/ml) | | | | | | | | |
| Titer (copies/μl) | | | | | | 4.5 × 10⁹ | 4.6 × 10⁹ | 1.6 × 10¹⁰ |

### Q5 PCR of Viral DNA

Conditions, as above, except: 50 ng viral DNA/50 μl assay, 20 PCR cycles 5 P.I out of 50
1. Loop I, primers A3CL-F+VR-I_-W-R, template A, size 644 bp
2. Loop IV, primers VR-I_IV-F+VR-IV_V-R, template B, size 145 bp
3. Loops V+VI, primers VR-IV_V-F+VR-VI_VII-R, tempi. A, size 194 bp
4. Loop VII, primers VR-VI_VR-VII-F+AXCL-R, template C, size 274 bp
Remaining 45 μl were purified using preparative gel, all four gel cutouts were pooled in one tube and purified using one column, final volume 50 μl H₂O.

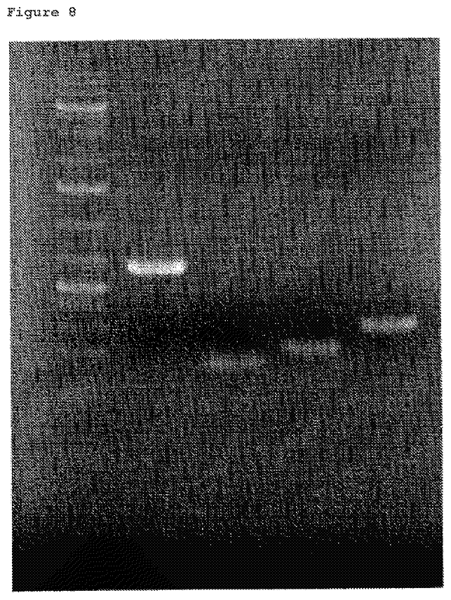

See FIG. 8.

### Overlap Extension

Full-length fragment was assembled without primers, substituting H₂O for the primers' volumes (5 pd) and subjected to the following overlap extension:

| | |
|---|---|
| 50 μl: | 10 μl 5xB Q5 |
| | 0.4 μl 25 mM dNTPs |
| | 25 μl (out of 50 μl) individual |
| | overlap 4 fragments mix (p. 12) |
| | 0.5 μl Q5 Pol |
| | 14.1 μl H₂O |
| 98° C. 30 sec | 15 cycles |
| 98° C. 10 sec | |
| 65° C. 20 sec | |
| 72° C. 60 sec | |
| 72° C. 2 min | |

After primer-less extension, the assay was split into 2×25 μl assays supplemented with A3CL-F, and A3CL-R primers, DNTPs, and fresh Q5, total volume 50 μl each.

### Assays are Subjected to the Following PCRs:

| | |
|---|---|
| 98° C. 30 sec | 20 cycles |
| 98° C. 10 sec | |
| 59° C. 20 sec | |
| 72° C. 60 sec | |
| 72° C. 2 min | |

ABC fragment was eluted in 50 μl, concentration 60 ng/μl (0.085 pmoles/μl).
D fragment was eluted in 50° C., concentration 46 ng/μl (0.065 pmoles/μl).

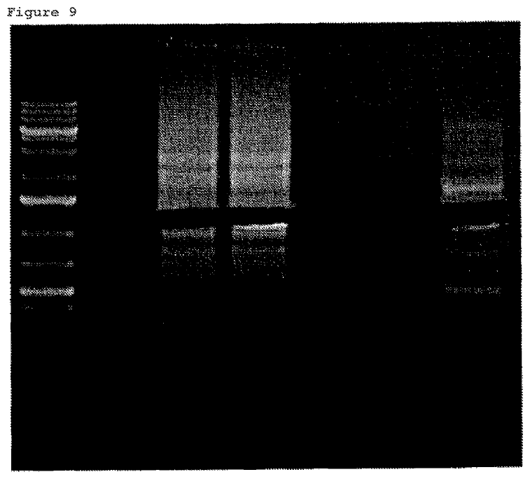

See FIG. 9.

### IDA Using NE Builder Master Mix

Total volume—200 μl, plasmid 1.5 μg (0.348 pmoles), insert—0.5 μg (0.7 pmoles), total DNA amount ˜1 pmole/200 μl assay.
Reaction 60 min @50° C. Lucigen electrocompetent E. coli cells, 8 ml, final density 0.8 A550. Library's complexity 2.5×10⁷.

1. A variant recombinant adeno-associated virus (rAAV) serotype 3B (AAV3B) capsid protein comprising the following set of substitutions:
(a) SNAX₁GTX₂X₃X₄X₅X₆LX₇ (SEQ ID NO: 7) in variable region (VR) IV, where X₁ is independently G or S; X₂ is A; X₃ is independently N or G; X₄ is independently T or M; X₅ is independently R or N; X₆ is independently R or G; and X₇ is M.

2. The variant of claim 1, further comprising each of the following sets of substitutions:
(b)
(SEQ ID NO: 4)
TIDTQNNNSNFPWTAASK in VR V;
(c)
(SEQ ID NO: 9)
KDDEER in VR VI;
and
(d)
(SEQ ID NO: 6)
QGAEASNVEVGK in VR VIII[[;]].

3. The variant of claim 1 comprising one or more of (a) SNAGGTANMNRLM (SEQ ID NO: 3) in VR IV, (b) TIDTQNNNSNFPWTAASK (SEQ ID NO: 4) in VR V, (c) KDDEER (SEQ ID NO: 9) in VR VI, and (d) QGAEASNVEVGK (SEQ ID NO: 6) in VR VIII.

4. The variant of claim 1 comprising (a) SNASGTAGTRGLM (SEQ ID NO: 8) in VR IV.

5. The variant of claim 1, wherein the capsid protein comprises an amino acid sequence that is at least 95% identical to the sequence set forth in SEQ ID NO: 2.

6. The variant of claim 1, wherein the capsid protein comprises the amino acid sequence set forth in SEQ ID NO: 2.

7. The variant of claim 1, wherein the capsid protein comprises an amino acid sequence that is at least 95% identical to the sequence set forth in SEQ ID NO: 10.

8. The variant of claim 1, wherein the capsid protein comprises the amino acid sequence set forth in SEQ ID NO: 10.

9. A recombinant AAV particle comprising the variant recombinant AAV3B capsid protein of claim 1.

10. The recombinant AAV3B particle of claim 9, further comprising a nucleic acid comprising a transgene of interest.

11. The recombinant AAV3B particle of claim 10, wherein the transgene is SERPINA1 or TRR.

12. (canceled)

13. The recombinant AAV particle of claim 9, wherein the nucleic acid is single stranded or self-complementary.

14. (canceled)

15. A composition comprising a plurality of the variant recombinant AAV3B particle of claim 9.

16. The composition of claim 15, further comprising a pharmaceutically acceptable carrier.

17. A method of transducing a hepatic cell with a transgene of interest, the method comprising providing to the hepatic cell the recombinant AAV particle of claim 9.

18. A method of treating a disease or disorder comprising administering the recombinant AAV particle of claim 9 to a subject in need thereof.

19. The method of claim 18, wherein the disease or disorder is Alpha-1 Antitrypsin Deficiency or Transthyretin-Related Familial Amyloid Polyneuropathy.

20. (canceled)

21. The method of claim 18, wherein the step of administering provides about a 15%, a 30%, a 50%, a 100%, a 200%, a 300%, a 400%, a 500%, a 750%, or a 1000% increase in transduction of the transgene of interest in hepatic cells in the subject, relative to a wild-type recombinant AAV3B particle.

22. The method of claim 18, wherein the subject is a primate.

23. The method of claim 18, wherein the subject is human.

24.-25. (canceled)


SEQUENCE LISTING (357 sequences)

SEQ ID NO: 1 - protein, 736 aa, unidentified
  source 1..736: note = adeno-associated virus serotype 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 2 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSASGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQSNASGTAGTRGMKFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 3 - protein, 13 aa, synthetic construct
SNAGGTANMNRLM

SEQ ID NO: 4 - protein, 18 aa, synthetic construct
TIDTQNNNSNFPWTAASK

SEQ ID NO: 5 - DNA, 1140 bp, synthetic construct
  source 1..1140: mol_type = other DNA
accaccagcaccagaacctgggccctgcccacttacaacaaccatctctacaagcaaatctccagcvvmdcaggagctascaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaargcamcvcnrgcggaacarccrvcmhsmrsvvsctgvngtttagccaggctgggcctcagtctatgtctttgcaggccagaaattggctacctgggccctgctaccggcaacagagactttcaamarycbmcrvcsrsaacaacaacagtrastttccttggmcagcggccagcamatatcatctcaatggccgcgactcgctggtgaatccaggaccagctatggccagtcacrrggacgatrmsgrsaratttttccctatgcacggcaatctaatatttggcaaasaarrcrscrvsrvarvcratrycgmsdwcgrsvrsgtaatgattacggatgaagaagagattcgtaccaccaatcctgtggcaacagagcagtatggaactgtggcaaataacttgcagrvsvvsmrsrvcvvscccacgdhtvvsrnsgtcvmscatcagggggccttacctggcatggtgtggcaagatcgtgacgtctaccttcaaggacctatctgggca

SEQ ID NO: 6 - protein, 12 aa, synthetic construct
QGAEASNVEVGK

SEQ ID NO: 7 - protein, 13 aa, synthetic construct
  VARIANT 4: note = may be G or S
  VARIANT 8: note = may be N or G
  VARIANT 9: note = may be T or M
  VARIANT 10: note = may be R or N
  VARIANT 11: note = may be R or G
SNAXGTAXXXXLM

SEQ ID NO: 8 - protein, 13 aa, synthetic construct
SNASGTAGTRGLM

SEQ ID NO: 9 - protein, 6 aa, synthetic construct
KDDEER

SEQ ID NO: 10 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSASGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQSNAGGTANMNRLMFSQAGPQSMSLQARNWLPGPCYRQQRLSTIDTQNNNSNFPWTAASKYHLNGRDSLVNPGPAMASHKDDEERFFPMHGNLIFGKQGAEASNVEVGKVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 11 - protein, 496 aa, unidentified
  source 1..496: note = adeno-associated virus serotype 3B
TTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 12 - DNA, 1491 bp, unidentified
  source 1..1491: mol_type = genomic DNA; note = adeno-associated virus serotype 3B
accaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtctaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 13 - DNA, 1491 bp, synthetic construct
  source 1..1491: mol_type = other DNA
accaccagcaccagaacctgggccctgcccacttacaacaaccatctctacaagcaaatctccagccaatcaggagctascaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaargcamcvcnrgcggaacarccrvcmhsmrsvvsctgvngtttagccaggctgggcctcagtctatgtctttgcaggccagaaattggctacctgggccctgctaccggcaacagagactttcaamarycbmcrvcsrsaacaacaacagtrastttccttggmcagcggccagcamatatcatctcaatggccgcgactcgctggtgaatccaggaccagctatggccagtcacrrggacgatrmsgrsaratttttccctatgcacggcaatctaatatttggcaaasaarrcrscrvsrvarvcratrycgmsdwcgrsvrsgtaatgattacggatgaagaagagattcgtaccaccaatcctgtggcaacagagcagtatggaactgtggcaaataacttgcagrvsvvsmrsrvcvvscccacgdhtvvsrnsgtcvmscatcagggggccttacctggcatggtgtggcaagatcgtgacgtctaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 14 - protein, 620 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSXXGAXNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQXXXXGTXXXXXLXFSQAGPQSMSLQARNWLPGPCYRQQRLSXXXXXNNNSXFPWXAASXYHLNGRDSLVNPGPAMASHXDDXXXFFPMHGNLIFGKXXXXXXXXXXXXVMITDEEEIRTTNPVATEQYGTVANNLQXXXXXPTXXXVXHQGALPGMVWQDRDVYLQGPIWA

SEQ ID NO: 15 - protein, 12 aa, synthetic construct
  VARIANT 5: note = may be Q, N, K, T, S, R, H, P, D, E, A, or G
  VARIANT 6: note = may be S, T, or A
  VARIANT 9: note = may be S or T
QISSXXGAXNDN

SEQ ID NO: 16 - protein, 21 aa, synthetic construct
  VARIANT 6: note = may be G or S
  VARIANT 7: note = may be T or N
  VARIANT 8: note = may be T, P, or A
  VARIANT 9: note = may be S or G
  VARIANT 12: note = may be T or A
  VARIANT 13: note = may be N, T, S, D, A, or G
  VARIANT 14: note = may be Q, H, P, L, K, N, T, M, or I
  VARIANT 15: note = may be S, Q, H, R, K, or N
  VARIANT 16: note = may be R, K, N, T, S, Q, H, P, E, D, A, or G
  VARIANT 18: note = may be L, K, T, R, M, Q, P, E, A, G, or V
LNRTQXXXXGTXXXXXLXFSQ

SEQ ID NO: 17 - protein, 76 aa, synthetic construct
  VARIANT 7: note = may be K or T
  VARIANT 8: note = may be T, I, A, or V
  VARIANT 9: note = may be A, P, H, D, S, or Y
  VARIANT 10: note = may be N, T, S, D, A, or G
  VARIANT 11: note = may be D, E, G, Q, H, or R
  VARIANT 16: note = may be N, K, E, or D
  VARIANT 20: note = may be T or P
  VARIANT 24: note = may be K or T
  VARIANT 44: note = may be K, R, E, or G
  VARIANT 47: note = may be E, T, K, N, A, or D
  VARIANT 48: note = may be E, D, or G
  VARIANT 48: note = may be K or R
  VARIANT 62: note = may be E or Q
  VARIANT 63: note = may be G, N, S, or D
  VARIANT 64: note = may be T, S, G, or A
  VARIANT 65: note = may be T, K, N, R, S, E, D, A, or G
  VARIANT 66: note = may be A, K, T, R, E, or G
  VARIANT 67: note = may be S, N, T, D, A, or G
  VARIANT 68: note = may be N or D
  VARIANT 69: note = may be A, T, I, or V
  VARIANT 70: note = may be E, A, or D
  VARIANT 71: note = may be L, N, I, D, V, Y, or F
  VARIANT 72: note = may be D, E, or G
  VARIANT 73: note = may be N, K, R, S, Q, H, E, D, or G
RQQRLSXXXXXNNNSXFPWXAASXYHLNGRDSLVNPGPAMASHXDDXXXFFPMHGNLIFGKXXXXXXXXXXXXVMI

SEQ ID NO: 18 - protein, 21 aa, synthetic construct
  VARIANT 7: note = may be S, K, N, T, R, E, D, A, or G
  VARIANT 8: note = may be S, K, N, T, R, Q, H, P, E, D, A, or G
  VARIANT 9: note = may be N, Q, H, R, K, or S
  VARIANT 10: note = may be T, N, S, D, A, or G
  VARIANT 11: note = may be A, K, N, T, R, S, Q, H, P, E, D, or G
  VARIANT 14: note = may be T, N, I, D, A, V, Y, S, or F
  VARIANT 15: note = may be G, K, N, T, R, S, Q, H, P, E, D, or A
  VARIANT 16: note = may be T, K, N, R, S, M, I, E, D, A, G, or V
  VARIANT 18: note = may be N, T, K, P, Q, H, A, E, or D
VANNLQXXXXXPTXXXVXHQG

SEQ ID NO: 19 - protein, 17 aa, synthetic construct
  VARIANT 1: note = may be I or T
  VARIANT 2: note = may be A or P
  VARIANT 3: note = may be N, S, or G
  VARIANT 4: note = may be D or Q
  VARIANT 17: note = may be K or T
XXXXNNNSNFPWTAASX

SEQ ID NO: 20 - DNA, 20 bp, synthetic construct
  source 1..20: mol_type = other DNA
ggctgggcgacagagtcatc

SEQ ID NO: 21 - DNA, 24 bp, synthetic construct
  source 1..24: mol_type = other DNA
gctggagatttgcttgtagagatg

SEQ ID NO: 22 - DNA, 60 bp, synthetic construct
  source 1..60: mol_type = other DNA
catctctacaagcaaatctccagcvvmdcaggagctascaacgacaaccactactttggc

SEQ ID NO: 23 - DNA, 56 bp, synthetic construct
  source 1..56: mol_type = other DNA
ccaaggaaastyactgttgttgttsysgbygkvgrytkttgaaagtctctgttgcc

SEQ ID NO: 24 - DNA, 52 bp, synthetic construct
  source 1..52: mol_type = other DNA
aacaacaacagtrastttccttggmcagcggccagcamatatcatctcaatg

SEQ ID NO: 25 - DNA, 58 bp, synthetic construct
  source 1..58: mol_type = other DNA
gattgccgtgcatagggaaaaatytsycskyatcgtccyygtgactggccatagctgg

SEQ ID NO: 26 - DNA, 23 bp, synthetic construct
  source 1..23: mol_type = other DNA
atttttccctatgcacggcaatc

SEQ ID NO: 27 - DNA, 22 bp, synthetic construct
  source 1..22: mol_type = other DNA
catccgtgtgaggaatctttgc

SEQ ID NO: 28 - DNA, 25 bp, synthetic construct
  source 1..25: mol_type = other DNA
ttgcgttctgttcaggtagtacaga

SEQ ID NO: 29 - DNA, 80 bp, synthetic construct
  source 1..80: mol_type = other DNA
ctgtactacctgaacagaacgcaargcamcvcnrgcggaacarccrvcmhsmrsvvsctgvngtttagccaggctgggcc

SEQ ID NO: 30 - DNA, 21 bp, synthetic construct
  source 1..21: mol_type = other DNA
tttgccaaatattagattgcc

SEQ ID NO: 31 - DNA, 80 bp, synthetic construct
  source 1..80: mol_type = other DNA
cggcaatctaatatttggcaaasaarrcrscrvsrvarvcratrycgmsdwcgrsvrsgtaatgattacggatgaagaag

SEQ ID NO: 32 - DNA, 23 bp, synthetic construct
  source 1..23: mol_type = other DNA
ctgcaagttatttgccacagttc

SEQ ID NO: 33 - DNA, 78 bp, synthetic construct
  source 1..78: mol_type = other DNA
gaactgtggcaaataacttgcagrvsvvsmrsrvcvvscccacgdhtvvsrnsgtcvmscatcagggggccttacctg

SEQ ID NO: 34 - DNA, 28 bp, synthetic construct
  source 1..28: mol_type = other DNA
cagtatctgtactacctgaacagaacgc

SEQ ID NO: 35 - DNA, 28 bp, synthetic construct
  source 1..28: mol_type = other DNA
gcgttctgttcaggtagtacagatactg

SEQ ID NO: 36 - DNA, 26 bp, synthetic construct
  source 1..26: mol_type = other DNA
cctgggccctgctaccggcaacagag

SEQ ID NO: 37 - DNA, 26 bp, synthetic construct
  source 1..26: mol_type = other DNA
ctctgttgccggtagcagggcccagg

SEQ ID NO: 38 - DNA, 28 bp, synthetic construct
  source 1..28: mol_type = other DNA
ccctatgcacggcaatctaatatttggc

SEQ ID NO: 39 - DNA, 28 bp, synthetic construct
  source 1..28: mol_type = other DNA
gccaaatattagattgccgtgcataggg

SEQ ID NO: 40 - protein, 14 aa, synthetic construct
  VARIANT 2: note = may be S or N
  VARIANT 3: note = may be S or G
  VARIANT 4: note = may be N or R
  VARIANT 5: note = may be T or D
  VARIANT 6: note = may be A or N
  VARIANT 9: note = may be T or F
  VARIANT 11: note = may be T or D
  VARIANT 13: note = may be N or Q
  VARIANT 14: note = may be H or D
QXXXXXPTXRXVXX

SEQ ID NO: 41 - protein, 13 aa, unidentified
  source 1..13: note = adeno-associated virus serotype 3B
GTTSGTTNQSRLL

SEQ ID NO: 42 - protein, 18 aa, unidentified
  source 1..18: note = adeno-associated virus serotype 3B
KTANDNNNSNFPWTAASK

SEQ ID NO: 43 - protein, 6 aa, unidentified
  source 1..6: note = adeno-associated virus serotype 3B
KDDEEK

SEQ ID NO: 44 - protein, 12 aa, unidentified
  source 1..12: note = adeno-associated virus serotype 3B
EGTTASNAELDN

SEQ ID NO: 45 - protein, 13 aa, synthetic construct
GNTGGTTSPSRLR

SEQ ID NO: 46 - protein, 18 aa, synthetic construct
KIYDRNNNSNFPWTAAST

SEQ ID NO: 47 - protein, 6 aa, synthetic construct
GDDTGR

SEQ ID NO: 48 - protein, 12 aa, synthetic construct
QGTGEGNVEVGK

SEQ ID NO: 49 - protein, 13 aa, synthetic construct
STTSGTTGQRKLA

SEQ ID NO: 50 - protein, 18 aa, synthetic construct
KAYGHNNNSDFPWPAAST

SEQ ID NO: 51 - protein, 6 aa, synthetic construct
GDDEDR

SEQ ID NO: 52 - protein, 12 aa, synthetic construct
QDSGENDVAIGR

SEQ ID NO: 53 - protein, 13 aa, synthetic construct
GTASGTTANSNLK

SEQ ID NO: 54 - protein, 18 aa, synthetic construct
KTYSDNNNSNFPWTAASK

SEQ ID NO: 55 - protein, 6 aa, synthetic construct
GDDDDR

SEQ ID NO: 56 - protein, 12 aa, synthetic construct
EGTDGANVEIDR

SEQ ID NO: 57 - protein, 13 aa, synthetic construct
GTPSGTAAHKTLE

SEQ ID NO: 58 - protein, 18 aa, synthetic construct
KTSAENNNSNFPWTAASK

SEQ ID NO: 59 - protein, 6 aa, synthetic construct
GDDAGR

SEQ ID NO: 60 - protein, 12 aa, synthetic construct
EDAEGGDAAIGG

SEQ ID NO: 61 - protein, 13 aa, synthetic construct
STAGGTATQKALT

SEQ ID NO: 62 - protein, 18 aa, synthetic construct
KVHAHNNNSNFPWTAASK

SEQ ID NO: 63 - protein, 6 aa, synthetic construct
EDDTGR

SEQ ID NO: 64 - protein, 12 aa, synthetic construct
QDATRSNVAFEE

SEQ ID NO: 65 - protein, 13 aa, synthetic construct
GNPSGTTGLRGLT

SEQ ID NO: 66 - protein, 18 aa, synthetic construct
TTDNENNNSNFPWPAASK

SEQ ID NO: 67 - protein, 6 aa, synthetic construct
KDDAGK

SEQ ID NO: 68 - protein, 12 aa, synthetic construct
QGTDGNNIAFGE

SEQ ID NO: 69 - protein, 13 aa, synthetic construct
GNTSGTTSKRPLM

SEQ ID NO: 70 - protein, 18 aa, synthetic construct
TTANENNNSNFPWTAAST

SEQ ID NO: 71 - protein, 6 aa, synthetic construct
EDDNER

SEQ ID NO: 72 - protein, 12 aa, synthetic construct
EDAKGTDTEFDR

SEQ ID NO: 73 - protein, 13 aa, synthetic construct
SNASGTTGIHQLK

SEQ ID NO: 74 - protein, 18 aa, synthetic construct
TAPDRNNNSEFPWTAASK

SEQ ID NO: 75 - protein, 6 aa, synthetic construct
EDDNGR

SEQ ID NO: 76 - protein, 12 aa, synthetic construct
QNGATADTEVER

SEQ ID NO: 77 - protein, 13 aa, synthetic construct
SNTGGTTAMRELE

SEQ ID NO: 78 - protein, 18 aa, synthetic construct
KAPNDNNNSKFPWTAAST

SEQ ID NO: 79 - protein, 6 aa, synthetic construct
EDDTGK

SEQ ID NO: 80 - protein, 12 aa, synthetic construct
ESTAETDVEDGR

SEQ ID NO: 81 - protein, 13 aa, synthetic construct
SNASGTAGLQRLK

SEQ ID NO: 82 - protein, 18 aa, synthetic construct
KIPDQNNNSNFPWTAASK

SEQ ID NO: 83 - protein, 6 aa, synthetic construct
KDDNGK

SEQ ID NO: 84 - protein, 12 aa, synthetic construct
QSGGAADIDNGN

SEQ ID NO: 85 - protein, 13 aa, synthetic construct
GNPSGTTAPHRLL

SEQ ID NO: 86 - protein, 18 aa, synthetic construct
TIHNGNNNSNFPWPAAST

SEQ ID NO: 87 - protein, 6 aa, synthetic construct
KDDDGR

SEQ ID NO: 88 - protein, 12 aa, synthetic construct
QDGGTSNIDIDG

SEQ ID NO: 89 - protein, 13 aa, synthetic construct
GTPSGTTDLRELA

SEQ ID NO: 90 - protein, 18 aa, synthetic construct
KIPNDNNNSNFPWTAAST

SEQ ID NO: 91 - protein, 6 aa, synthetic construct
EDDEDR

SEQ ID NO: 92 - protein, 13 aa, synthetic construct
SNPSGTANPRTLM

SEQ ID NO: 93 - protein, 18 aa, synthetic construct
KIDAHNNNSEFPWPAAST

SEQ ID NO: 94 - protein, 6 aa, synthetic construct
EDDNGK

SEQ ID NO: 95 - protein, 12 aa, synthetic construct
QSSTTGDVEDDD

SEQ ID NO: 96 - protein, 13 aa, synthetic construct
GNASGTTDTKRLT

SEQ ID NO: 97 - protein, 18 aa, synthetic construct
KASGGNNNSNFPWTAAST

SEQ ID NO: 98 - protein, 6 aa, synthetic construct
KDDDDK

SEQ ID NO: 99 - protein, 12 aa, synthetic construct
EGSNRDDAEVDR

SEQ ID NO: 100 - protein, 13 aa, synthetic construct
GNTGGTTDIRRLR

SEQ ID NO: 101 - protein, 18 aa, synthetic construct
KTHSENNNSEFPWTAAST

SEQ ID NO: 102 - protein, 6 aa, synthetic construct
KDDNER

SEQ ID NO: 103 - protein, 12 aa, synthetic construct
QDTRETDVAIDR

SEQ ID NO: 104 - protein, 13 aa, synthetic construct
GNASGTAGMRELM

SEQ ID NO: 105 - protein, 18 aa, synthetic construct
KAANHNNNSNFPWTAASK

SEQ ID NO: 106 - protein, 6 aa, synthetic construct
EDDEDK

SEQ ID NO: 107 - protein, 12 aa, synthetic construct
ESGSADDVAIGR

SEQ ID NO: 108 - protein, 13 aa, synthetic construct
SNTGGTATPKQLQ

SEQ ID NO: 109 - protein, 18 aa, synthetic construct
KASAHNNNSEFPWTAASK

SEQ ID NO: 110 - protein, 12 aa, synthetic construct
EGSTRNDIANEH

SEQ ID NO: 111 - protein, 13 aa, synthetic construct
SNTGGTANIKELT

SEQ ID NO: 112 - protein, 18 aa, synthetic construct
KTSNDNNNSNFPWTAAST

SEQ ID NO: 113 - protein, 6 aa, synthetic construct
RDDNDK

SEQ ID NO: 114 - protein, 12 aa, synthetic construct
QSASKNDIEYEQ

SEQ ID NO: 115 - protein, 13 aa, synthetic construct
SNAGGTTSNRELR

SEQ ID NO: 116 - protein, 18 aa, synthetic construct
TTSSQNNNSNFPWTAAST

SEQ ID NO: 117 - protein, 6 aa, synthetic construct
RDDDDR

SEQ ID NO: 118 - protein, 12 aa, synthetic construct
QDAGGNDVEVGD

SEQ ID NO: 119 - protein, 13 aa, synthetic construct
SNTSGTATTSALK

SEQ ID NO: 120 - protein, 18 aa, synthetic construct
KTYGHNNNSNFPWTAASK

SEQ ID NO: 121 - protein, 6 aa, synthetic construct
GDDTEK

SEQ ID NO: 122 - protein, 12 aa, synthetic construct
QGGSANNVEVES

SEQ ID NO: 123 - protein, 13 aa, synthetic construct
SNAGGTAATNRLL

SEQ ID NO: 124 - protein, 6 aa, synthetic construct
RDDEDK

SEQ ID NO: 125 - protein, 12 aa, synthetic construct
EGGEKGNVDIDR

SEQ ID NO: 126 - protein, 13 aa, synthetic construct
STPSGTTATKGLT

SEQ ID NO: 127 - protein, 18 aa, synthetic construct
TAHTGNNNSNFPWTAASK

SEQ ID NO: 128 - protein, 6 aa, synthetic construct
GDDDGK

SEQ ID NO: 129 - protein, 12 aa, synthetic construct
EGSTATDVAIGS

SEQ ID NO: 130 - protein, 13 aa, synthetic construct
GNTGGTTDLRRLM

SEQ ID NO: 131 - protein, 18 aa, synthetic construct
TTDNHNNNSEFPWPAAST

SEQ ID NO: 132 - protein, 6 aa, synthetic construct
GDDKGR

SEQ ID NO: 133 - protein, 12 aa, synthetic construct
ENGAKNDIAFEG

SEQ ID NO: 134 - protein, 13 aa, synthetic construct
STTSGTTTLKALQ

SEQ ID NO: 135 - protein, 18 aa, synthetic construct
KIPNRNNNSNFPWTAAST

SEQ ID NO: 136 - protein, 12 aa, synthetic construct
ENSKGANTEIDE

SEQ ID NO: 137 - protein, 13 aa, synthetic construct
GTTSGTTDPKDLV

SEQ ID NO: 138 - protein, 18 aa, synthetic construct
TTHGDNNNSDFPWTAAST

SEQ ID NO: 139 - protein, 12 aa, synthetic construct
EDTAADNVEFGR

SEQ ID NO: 140 - protein, 13 aa, synthetic construct
STAGGTTTIKDLV

SEQ ID NO: 141 - protein, 18 aa, synthetic construct
KVPDDNNNSKFPWTAASK

SEQ ID NO: 142 - protein, 6 aa, synthetic construct
EDDDER

SEQ ID NO: 143 - protein, 12 aa, synthetic construct
QDSGATNVEFGR

SEQ ID NO: 144 - protein, 13 aa, synthetic construct
GTTGGTTTMRKLG

SEQ ID NO: 145 - protein, 18 aa, synthetic construct
KVYGGNNNSNFPWTAAST

SEQ ID NO: 146 - protein, 6 aa, synthetic construct
EDDAER

SEQ ID NO: 147 - protein, 12 aa, synthetic construct
QSSGRNDVEYGD

SEQ ID NO: 148 - protein, 13 aa, synthetic construct
GNTGGTASTRRLT

SEQ ID NO: 149 - protein, 18 aa, synthetic construct
KIPDQNNNSNFPWPAASK

SEQ ID NO: 150 - protein, 6 aa, synthetic construct
EDDEGR

SEQ ID NO: 151 - protein, 12 aa, synthetic construct
QSAEKGDIEYGR

SEQ ID NO: 152 - protein, 13 aa, synthetic construct
GNTSGTTATHTLA

SEQ ID NO: 153 - protein, 18 aa, synthetic construct
KIHSRNNNSDFPWPAASK

SEQ ID NO: 154 - protein, 6 aa, synthetic construct
EDDAGK

SEQ ID NO: 155 - protein, 12 aa, synthetic construct
QGATAGNIDVEQ

SEQ ID NO: 156 - protein, 13 aa, synthetic construct
SNPGGTTSIRGLQ

SEQ ID NO: 157 - protein, 18 aa, synthetic construct
TIPNRNNNSNFPWPAAST

SEQ ID NO: 158 - protein, 6 aa, synthetic construct
RDDTDK

SEQ ID NO: 159 - protein, 12 aa, synthetic construct
QGGGAGNTDFDH

SEQ ID NO: 160 - protein, 13 aa, synthetic construct
STTSGTAAPRGLV

SEQ ID NO: 161 - protein, 18 aa, synthetic construct
TVYGHNNNSEFPWTAASK

SEQ ID NO: 162 - protein, 6 aa, synthetic construct
KDDEGR

SEQ ID NO: 163 - protein, 12 aa, synthetic construct
EGAGASNVAIEE

SEQ ID NO: 164 - protein, 13 aa, synthetic construct
GNASGTATKQGLM

SEQ ID NO: 165 - protein, 18 aa, synthetic construct
KVPNQNNNSDFPWTAASK

SEQ ID NO: 166 - protein, 6 aa, synthetic construct
KDDDDR

SEQ ID NO: 167 - protein, 12 aa, synthetic construct
QSSDKNNAEDDS

SEQ ID NO: 168 - protein, 13 aa, synthetic construct
SNAGGTATTSQLR

SEQ ID NO: 169 - protein, 18 aa, synthetic construct
TAPAENNNSNFPWPAAST

SEQ ID NO: 170 - protein, 6 aa, synthetic construct
RDDADR

SEQ ID NO: 171 - protein, 12 aa, synthetic construct
ESGRGDNVDFEK

SEQ ID NO: 172 - protein, 13 aa, synthetic construct
SNTGGTAGIRALQ

SEQ ID NO: 173 - protein, 18 aa, synthetic construct
KVDTGNNNSDFPWTAAST

SEQ ID NO: 174 - protein, 6 aa, synthetic construct
EDDTEK

SEQ ID NO: 175 - protein, 12 aa, synthetic construct
ENSARNDADIGR

SEQ ID NO: 176 - protein, 13 aa, synthetic construct
GNASGTAAQNGLR

SEQ ID NO: 177 - protein, 18 aa, synthetic construct
KIPNENNNSKFPWTAAST

SEQ ID NO: 178 - protein, 6 aa, synthetic construct
KDDEGK

SEQ ID NO: 179 - protein, 12 aa, synthetic construct
ESSSGDDAEFGG

SEQ ID NO: 180 - protein, 13 aa, synthetic construct
SNTSGTAGPQQLR

SEQ ID NO: 181 - protein, 18 aa, synthetic construct
KTHAQNNNSNFPWTAASK

SEQ ID NO: 182 - protein, 12 aa, synthetic construct
EGARANDIAFDQ

SEQ ID NO: 183 - protein, 13 aa, synthetic construct
STPSGTTSMRTLE

SEQ ID NO: 184 - protein, 18 aa, synthetic construct
KAPARNNNSEFPWTAAST

SEQ ID NO: 185 - protein, 6 aa, synthetic construct
RDDAGK

SEQ ID NO: 186 - protein, 12 aa, synthetic construct
QGSRENDTEFDG

SEQ ID NO: 187 - protein, 13 aa, synthetic construct
STAGGTTALKGLK

SEQ ID NO: 188 - protein, 18 aa, synthetic construct
TIADHNNNSEFPWPAAST

SEQ ID NO: 189 - protein, 6 aa, synthetic construct
RDDKEK

SEQ ID NO: 190 - protein, 12 aa, synthetic construct
EDSTGANIADDR

SEQ ID NO: 191 - protein, 13 aa, synthetic construct
STPSGTASTRTLM

SEQ ID NO: 192 - protein, 18 aa, synthetic construct
KTHNHNNNSEFPWTAAST

SEQ ID NO: 193 - protein, 6 aa, synthetic construct
KDDEDK

SEQ ID NO: 194 - protein, 12 aa, synthetic construct
EGTEATNVAIGG

SEQ ID NO: 195 - protein, 13 aa, synthetic construct
GNPGGTTNNQALR

SEQ ID NO: 196 - protein, 18 aa, synthetic construct
KIHGQNNNSDFPWTAAST

SEQ ID NO: 197 - protein, 12 aa, synthetic construct
EGSARGDVAYEK

SEQ ID NO: 198 - protein, 13 aa, synthetic construct
SNASGTTDTRELV

SEQ ID NO: 199 - protein, 18 aa, synthetic construct
TIADDNNNSEFPWTAAST

SEQ ID NO: 200 - protein, 12 aa, synthetic construct
QGSAGADVEVEK

SEQ ID NO: 201 - protein, 13 aa, synthetic construct
SNPGGTTNLRELR

SEQ ID NO: 202 - protein, 18 aa, synthetic construct
TIHTENNNSEFPWTAAST

SEQ ID NO: 203 - protein, 6 aa, synthetic construct
RDDKDR

SEQ ID NO: 204 - protein, 12 aa, synthetic construct
QGGGGTNVEIGS

SEQ ID NO: 205 - protein, 13 aa, synthetic construct
GTTGGTANNNTLL

SEQ ID NO: 206 - protein, 18 aa, synthetic construct
KIASGNNNSNFPWTAAST

SEQ ID NO: 207 - protein, 6 aa, synthetic construct
KDDKGR

SEQ ID NO: 208 - protein, 12 aa, synthetic construct
EGAEKNDTAVGN

SEQ ID NO: 209 - protein, 13 aa, synthetic construct
GTTSGTTDKQQLM

SEQ ID NO: 210 - protein, 18 aa, synthetic construct
KTHNGNNNSDFPWTAASK

SEQ ID NO: 211 - protein, 12 aa, synthetic construct
QSAEGNNVAYDG

SEQ ID NO: 212 - protein, 13 aa, synthetic construct
GTAGGTATLSTLV

SEQ ID NO: 213 - protein, 18 aa, synthetic construct
KISAGNNNSDFPWTAASK

SEQ ID NO: 214 - protein, 6 aa, synthetic construct
GDDNGK

SEQ ID NO: 215 - protein, 12 aa, synthetic construct
QNSTASDVAIDG

SEQ ID NO: 216 - protein, 13 aa, synthetic construct
SNTSGTAGLRTLT

SEQ ID NO: 217 - protein, 18 aa, synthetic construct
KADADNNNSDFPWPAAST

SEQ ID NO: 218 - protein, 12 aa, synthetic construct
EDASGNNVEDGR

SEQ ID NO: 219 - protein, 13 aa, synthetic construct
SNASGTATPSTLR

SEQ ID NO: 220 - protein, 18 aa, synthetic construct
KTDTHNNNSEFPWPAASK

SEQ ID NO: 221 - protein, 6 aa, synthetic construct
RDDNDR

SEQ ID NO: 222 - protein, 12 aa, synthetic construct
EGARGSNIDVGD

SEQ ID NO: 223 - protein, 13 aa, synthetic construct
STASGTASLRALM

SEQ ID NO: 224 - protein, 18 aa, synthetic construct
KVPNRNNNSNFPWTAAST

SEQ ID NO: 225 - protein, 6 aa, synthetic construct
GDDNDK

SEQ ID NO: 226 - protein, 12 aa, synthetic construct
ENARASDAEVDR

SEQ ID NO: 227 - protein, 13 aa, synthetic construct
GTASGTATTKGLL

SEQ ID NO: 228 - protein, 18 aa, synthetic construct
KISTQNNNSNFPWTAASK

SEQ ID NO: 229 - protein, 6 aa, synthetic construct
EDDADK

SEQ ID NO: 230 - protein, 12 aa, synthetic construct
QGGETDNVDVGD

SEQ ID NO: 231 - protein, 13 aa, synthetic construct
GTASGTAALKQLA

SEQ ID NO: 232 - protein, 18 aa, synthetic construct
KADSDNNNSNFPWTAAST

SEQ ID NO: 233 - protein, 6 aa, synthetic construct
EDDADR

SEQ ID NO: 234 - protein, 12 aa, synthetic construct
QGGETGNIEYDG

SEQ ID NO: 235 - protein, 13 aa, synthetic construct
GNASGTATTSNLM

SEQ ID NO: 236 - protein, 18 aa, synthetic construct
KADDRNNNSNFPWTAAST

SEQ ID NO: 237 - protein, 6 aa, synthetic construct
KDDEDR

SEQ ID NO: 238 - protein, 12 aa, synthetic construct
QGAKRSDTAVEE

SEQ ID NO: 239 - protein, 13 aa, synthetic construct
GTAGGTANMKDLR

SEQ ID NO: 240 - protein, 18 aa, synthetic construct
TTASENNNSDFPWTAAST

SEQ ID NO: 241 - protein, 6 aa, synthetic construct
EDDKDK

SEQ ID NO: 242 - protein, 12 aa, synthetic construct
EGANGGDVAIGQ

SEQ ID NO: 243 - protein, 13 aa, synthetic construct
STPGGTTTIRDLK

SEQ ID NO: 244 - protein, 18 aa, synthetic construct
TVSTDNNNSDFPWPAAST

SEQ ID NO: 245 - protein, 12 aa, synthetic construct
EGSGRNNVAVEE

SEQ ID NO: 246 - protein, 13 aa, synthetic construct
STPSGTANINTLR

SEQ ID NO: 247 - protein, 18 aa, synthetic construct
KTPNRNNNSNFPWTAAST

SEQ ID NO: 248 - protein, 12 aa, synthetic construct
QSATKDDVDIGG

SEQ ID NO: 249 - protein, 13 aa, synthetic construct
GNTGGTAGLQKLM

SEQ ID NO: 250 - protein, 18 aa, synthetic construct
KTHGGNNNSNFPWPAAST

SEQ ID NO: 251 - protein, 6 aa, synthetic construct
KDDDGK

SEQ ID NO: 252 - protein, 12 aa, synthetic construct
QSSRGNDVAVDD

SEQ ID NO: 253 - protein, 13 aa, synthetic construct
GNTSGTTTPRTLA

SEQ ID NO: 254 - protein, 18 aa, synthetic construct
KIPSHNNNSEFPWTAASK

SEQ ID NO: 255 - protein, 12 aa, synthetic construct
QGSNGSNIEFGS

SEQ ID NO: 256 - protein, 13 aa, synthetic construct
SNAGGTAGLRQLT

SEQ ID NO: 257 - protein, 18 aa, synthetic construct
KAPAENNNSDFPWTAAST

SEQ ID NO: 258 - protein, 12 aa, synthetic construct
EGGGGANIAVEE

SEQ ID NO: 259 - protein, 13 aa, synthetic construct
GTASGTAAKSTLV

SEQ ID NO: 260 - protein, 18 aa, synthetic construct
KISTRNNNSNFPWTAAST

SEQ ID NO: 261 - protein, 6 aa, synthetic construct
EDDEEK

SEQ ID NO: 262 - protein, 12 aa, synthetic construct
EGTSKNDVEVEN

SEQ ID NO: 263 - protein, 13 aa, synthetic construct
GTTSGTTTTRRLM

SEQ ID NO: 264 - protein, 18 aa, synthetic construct
KIYGGNNNSNFPWTAAST

SEQ ID NO: 265 - protein, 6 aa, synthetic construct
EDDAGR

SEQ ID NO: 266 - protein, 12 aa, synthetic construct
QGTATANVEVES

SEQ ID NO: 267 - protein, 13 aa, synthetic construct
GTAGGTAGMRELA

SEQ ID NO: 268 - protein, 18 aa, synthetic construct
TIYTGNNNSNFPWTAASK

SEQ ID NO: 269 - protein, 12 aa, synthetic construct
EGSSTGDADVGR

SEQ ID NO: 270 - protein, 13 aa, synthetic construct
STTGGTANPKELR

SEQ ID NO: 271 - protein, 18 aa, synthetic construct
TAANHNNNSEFPWTAAST

SEQ ID NO: 272 - protein, 12 aa, synthetic construct
QGAGESNVAIDG

SEQ ID NO: 273 - protein, 13 aa, synthetic construct
GTTSGTTGTSTLR

SEQ ID NO: 274 - protein, 18 aa, synthetic construct
KTDTGNNNSNFPWTAASK

SEQ ID NO: 275 - protein, 6 aa, synthetic construct
GDDEER

SEQ ID NO: 276 - protein, 12 aa, synthetic construct
EGAGTADAAVDG

SEQ ID NO: 277 - protein, 13 aa, synthetic construct
GNAGGTTNKRDLL

SEQ ID NO: 278 - protein, 18 aa, synthetic construct
TAYTRNNNSDFPWTAAST

SEQ ID NO: 279 - protein, 6 aa, synthetic construct
KDDDEK

SEQ ID NO: 280 - protein, 12 aa, synthetic construct
QGTGKTDADNGG

SEQ ID NO: 281 - protein, 13 aa, synthetic construct
GNASGTTDMKHLT

SEQ ID NO: 282 - protein, 18 aa, synthetic construct
KISDRNNNSNFPWPAAST

SEQ ID NO: 283 - protein, 6 aa, synthetic construct
RDDNER

SEQ ID NO: 284 - protein, 12 aa, synthetic construct
QSTRGGNAEIDG

SEQ ID NO: 285 - protein, 13 aa, synthetic construct
SNTGGTADLRDLL

SEQ ID NO: 286 - protein, 18 aa, synthetic construct
TIPTQNNNSEFPWTAASK

SEQ ID NO: 287 - protein, 6 aa, synthetic construct
KDDNDR

SEQ ID NO: 288 - protein, 12 aa, synthetic construct
QSAKANDVEVDR

SEQ ID NO: 289 - protein, 13 aa, synthetic construct
STASGTTATQQLV

SEQ ID NO: 290 - protein, 18 aa, synthetic construct
TTDSQNNNSEFPWTAAST

SEQ ID NO: 291 - protein, 12 aa, synthetic construct
ENAEGGNVEIGQ

SEQ ID NO: 292 - protein, 13 aa, synthetic construct
GNAGGTTANKTLM

SEQ ID NO: 293 - protein, 18 aa, synthetic construct
KIAAHNNNSEFPWTAAST

SEQ ID NO: 294 - protein, 6 aa, synthetic construct
RDDDGK

SEQ ID NO: 295 - protein, 12 aa, synthetic construct
QDSSADNIEYGK

SEQ ID NO: 296 - protein, 13 aa, synthetic construct
GNAGGTTGTKELR

SEQ ID NO: 297 - protein, 18 aa, synthetic construct
TIANENNNSNFPWTAAST

SEQ ID NO: 298 - protein, 12 aa, synthetic construct
EDAKRNNVDYDG

SEQ ID NO: 299 - protein, 13 aa, synthetic construct
SNPSGTTGKSSLK

SEQ ID NO: 300 - protein, 18 aa, synthetic construct
KTSNENNNSNFPWTAASK

SEQ ID NO: 301 - protein, 12 aa, synthetic construct
EDSRAGDADFEK

SEQ ID NO: 302 - protein, 13 aa, synthetic construct
GNTSGTASIRQLQ

SEQ ID NO: 303 - protein, 18 aa, synthetic construct
KTPDGNNNSKFPWTAASK

SEQ ID NO: 304 - protein, 6 aa, synthetic construct
RDDNGR

SEQ ID NO: 305 - protein, 12 aa, synthetic construct
EGSTEGNIEIEG

SEQ ID NO: 306 - protein, 13 aa, synthetic construct
GNTGGTTTLSALG

SEQ ID NO: 307 - protein, 18 aa, synthetic construct
TAHTQNNNSNFPWTAASK

SEQ ID NO: 308 - protein, 12 aa, synthetic construct
QSSTGGDTAFDG

SEQ ID NO: 309 - protein, 13 aa, synthetic construct
SNPSGTTTTQRLQ

SEQ ID NO: 310 - protein, 18 aa, synthetic construct
KTDNDNNNSDFPWPAAST

SEQ ID NO: 311 - protein, 6 aa, synthetic construct
RDDTDR

SEQ ID NO: 312 - protein, 12 aa, synthetic construct
EGSTGGDAEIER

SEQ ID NO: 313 - protein, 13 aa, synthetic construct
SNASGTTTMRKLG

SEQ ID NO: 314 - protein, 18 aa, synthetic construct
TISSGNNNSNFPWPAASK

SEQ ID NO: 315 - protein, 6 aa, synthetic construct
GDDNEK

SEQ ID NO: 316 - protein, 12 aa, synthetic construct
QDSSENDVADER

SEQ ID NO: 317 - protein, 13 aa, synthetic construct
STAGGTATMQRLM

SEQ ID NO: 318 - protein, 18 aa, synthetic construct
TTDTGNNNSNFPWTAAST

SEQ ID NO: 319 - protein, 12 aa, synthetic construct
QGGEGGDIEDDR

SEQ ID NO: 320 - protein, 13 aa, synthetic construct
GTAGGTATTRDLQ

SEQ ID NO: 321 - protein, 18 aa, synthetic construct
TTDDHNNNSDFPWTAASK

SEQ ID NO: 322 - protein, 6 aa, synthetic construct
KDDNGR

SEQ ID NO: 323 - protein, 12 aa, synthetic construct
QGGRGANTAYEG

SEQ ID NO: 324 - protein, 13 aa, synthetic construct
GTTGGTAAMSALR

SEQ ID NO: 325 - protein, 18 aa, synthetic construct
TTDDGNNNSKFPWPAASK

SEQ ID NO: 326 - protein, 12 aa, synthetic construct
EDGGTSNAAIGD

SEQ ID NO: 327 - protein, 13 aa, synthetic construct
GNTSGTTTNRELM

SEQ ID NO: 328 - protein, 12 aa, synthetic construct
EDTGRADVEVGR

SEQ ID NO: 329 - protein, 13 aa, synthetic construct
SNAGGTADKQDLV

SEQ ID NO: 330 - protein, 18 aa, synthetic construct
TAHSENNNSNFPWTAASK

SEQ ID NO: 331 - protein, 6 aa, synthetic construct
EDDDDR

SEQ ID NO: 332 - protein, 12 aa, synthetic construct
QGAAGGDIEVGS

SEQ ID NO: 333 - protein, 13 aa, synthetic construct
GNASGTAATHELL

SEQ ID NO: 334 - protein, 18 aa, synthetic construct
TTHDHNNNSDFPWTAASK

SEQ ID NO: 335 - protein, 6 aa, synthetic construct
RDDAER

SEQ ID NO: 336 - protein, 12 aa, synthetic construct
EGGAKSDVDFGS

SEQ ID NO: 337 - protein, 13 aa, synthetic construct
SNASGTADTRHLM

SEQ ID NO: 338 - protein, 18 aa, synthetic construct
TTPGENNNSDFPWPAAST

SEQ ID NO: 339 - protein, 6 aa, synthetic construct
GDDTGK

SEQ ID NO: 340 - protein, 12 aa, synthetic construct
QGSATTDIEYGE

SEQ ID NO: 341 - protein, 13 aa, synthetic construct
SNTSGTTAQSKLQ

SEQ ID NO: 342 - protein, 18 aa, synthetic construct
KIHNRNNNSDFPWTAAST

SEQ ID NO: 343 - protein, 12 aa, synthetic construct
QGAEGSDVAVGD

SEQ ID NO: 344 - protein, 13 aa, synthetic construct
GNPSGTADQRALQ

SEQ ID NO: 345 - protein, 18 aa, synthetic construct
KIPTGNNNSDFPWTAAST

SEQ ID NO: 346 - protein, 6 aa, synthetic construct
RDDTEK

SEQ ID NO: 347 - protein, 12 aa, synthetic construct
QGTGGSDIEIGG

SEQ ID NO: 348 - protein, 13 aa, synthetic construct
SNASGTAGLNALK

SEQ ID NO: 349 - protein, 18 aa, synthetic construct
KAYTHNNNSDFPWTAAST

SEQ ID NO: 350 - protein, 6 aa, synthetic construct
GDDDER

SEQ ID NO: 351 - protein, 12 aa, synthetic construct
ENAKAGNAAIDG

SEQ ID NO: 352 - protein, 13 aa, synthetic construct
GNPSGTTNLQRLM

SEQ ID NO: 353 - protein, 18 aa, synthetic construct
KIDDQNNNSNFPWPAAST

SEQ ID NO: 354 - protein, 12 aa, synthetic construct
ESGGTADVAVDK

SEQ ID NO: 355 - protein, 13 aa, synthetic construct
GTTSGTASIQRLQ

SEQ ID NO: 356 - protein, 18 aa, synthetic construct
KTYADNNNSEFPWTAAST

SEQ ID NO: 357 - protein, 12 aa, synthetic construct
ESAAGSDTEVDG